(19) United States
(12) Patent Application Publication
(10) Pub. No.: US 2018/0298379 A1
(43) Pub. Date: Oct. 18, 2018

(54) SMALL INTERFERING RNA MODIFICATION METHOD BY COMBINING WITH ISONUCLEOSIDE MODIFICATION, TERMINAL PEPTIDE CONJUGATION AND CATIONIC LIPOSOMES, AND PREPARATION

(71) Applicant: PEKING UNIVERSITY, Beijing (CN)

(72) Inventors: Zhenjun YANG, Beijing (CN); Jing SUN, Beijing (CN); Xinmeng FAN, Beijing (CN); Xiaogeng WANG, Beijing (CN); Ye HUANG, Beijing (CN); Jiancheng WANG, Beijing (CN); Chong QUI, Beijing (CN)

(21) Appl. No.: 15/735,576

(22) PCT Filed: Jun. 12, 2015
(86) PCT No.: PCT/CN2015/000402

§ 371 Date: Dec. 11, 2017

(51) Int. Cl. C12N 15/11; C12N 15/113

(52) U.S. Cl. CPC C12N 15/111; C12N 15/1137; C12N 2320/32; C12N 2310/3513; C12N 2310/3515; C12N 2310/14

(57) ABSTRACT

Provided is a novel chemical modification method for small interfering RNA (siRNA). The method is combined with at least two of the three methods of isonucleoside modification, terminal peptide conjugation and cationic liposomes.

### RELATED APPLICATIONS

This application, filed under 35 U.S.C. § 371, is a U.S. national phase application of and based on PCT/CN2015/000402, filed Jun. 12, 2015, which is hereby incorporated by reference in its entirety.

### TECHNICAL FIELD

Embodiments relate to the technical field of biomedicine. More specifically, embodiments of the invention relate to an integrated siRNA chemical modification method.

### BACKGROUND

RNA interference (RNAi) was first discovered by Nobel laureate Fire and co-workers in Caenorhabditiselegans in 1998, and was first discovered in mammalian cells in 2001. The RNAi phenomenon is currently considered as a conserved mechanism of body defense evolved in the living body and is present in all kinds of animals. Chemically synthesized siRNAs typically consist of two 19-22 nt single-stranded RNAs complementary to each other. Typically, the 3′-end of the strands do not participate in pairing and are referred to as 3′-overhang. RNA sequences that target mRNA are designed as sequences that are completely complementary to the mRNA, are referred to as the guide strand or the antisense strand, and the other strand is the same as the mRNA sequence, known as the passenger strand or sense strand. Typically, a synthetic 21-nt siRNA enters the cell and is firstly phosphorylated by the Clp1 enzyme at its 5′-end. After that, it is recognized by the TRBP protein and undergoes a series of processes to form unactivated RISC complexes with Dicer, Ago 2 and TRBP proteins. As a result of cleavage of sense strand by Ago 2 and its subsequent departure, an activated RISC complex will form. Finally, the mRNA complementary to the guide strand is loaded into the RISC and undergoes passenger strand-like endonuclease degradation to cause a gene silencing effect.

However, to develop siRNA technology into producing ideal drugs, following requirements have to be met: (1) siRNAs must have high specificity and affinity for the target sequence to reduce immunogenicity caused by off-target effects; (2) they must have high in vivo stability, resistant to degradation of plasma nuclease; (3) they must have good cell membrane permeability in order to achieve effective cellular uptake; (4) they must be efficient access to the cytoplasm (where RNAi play a role) to reduce their destruction effect due to degradation by enzymes and other substances within the cell. However, siRNAs composed of natural bases can not meet these requirements and must be modified by means of covalent or non-covalent chemical modifications, so as to improve the prospect of turning siRNA into drugs. At the present, commonly used means of covalent chemical modifications include: sugar ring-, base-, phosphoric acid skeleton-, terminal-modifications and the like; whereas non-covalent modification mainly means adoption of vectors possessing cationic characteristics to achieve the inclusion and delivery of siRNAs. However, a single chemical modification method often has some limitations, such as: to covalently conjugate the siRNA itself and modify their bases can improve the stability of siRNA and its selectivity to the target mRNA, thereby reducing off-target effect to some extent, yet these can not achieve effective cell uptake of resulting siRNA; although non-covalent binding of the vector using siRNA to encapsulate them do solve the big problem of cellular uptake, and be able to enhance the transmembrane transport ability of siRNA to some extent, yet this single siRNA/vector electrostatic composites can not achieve high siRNA silencing efficiency due to their random way of entering the cell, in addition, too much cationic material will bring about some cytotoxic side effects. Therefore, in order to thoroughly and systematically solve various problems existing in natural siRNA, the present invention attempts to adopt a combination of multiple chemical modification strategies to achieve the safety and high efficiency of siRNA transfection.

### SUMMARY

In order to overcome the shortcomings of siRNA in researches of clinical applications, the present invention provides a novel chemical modification method of siRNA. The method provided by the present invention is a joint employment of two or all of the following three modifications: isonucleoside modification, terminal peptide conjugation and cationic lipids vector encapsulation. The product obtained by this joint chemical modification methods has the advantages of stable physical and chemical properties, good and controllable biological behaviors, highly efficient bio-activities, and so on, and can be widely used in the researches of anti-virus and anti-tumor drugs.

Embodiments of the invention closely integrates two strategies (structural modification and vector transport) commonly used in the current application of siRNA by deliberate assembling to form a controllable supramolecular complex system, so as to realize the regulation and control of the biological behavior of siRNA (endocytosis pathway and intracellular metabolism), making it to play an effective role in silencing. Therefore, the present invention is intended to guide the biological behavior of the siRNA/vector composite by regulating the assembly structure of the siRNA/vector composite, and finally to realize the high efficiency of siRNA. This also helps to reduce the dose of siRNA and its associated vectors and further avoids or reduces its side effects such as toxicity or immunogenicity.

An integrated chemical modification of a siRNA of the present invention comprises: incorporation of a D- or an L-isonucleoside at one or more sites of the sense and/or antisense strands of a siRNA; bispeptide conjugation modifications at the 3′-end of the sense and/or antisense strands of siRNAs; as well as encapsulating the siRNAs with gemini type cationic liposome vectors. By modification with two or all of the above ways, this method can effectively improve the stability of the siRNA, reduce the off-target effect and regulate and control its transmembrane pathways, so that the siRNA can enter the cells according to the desired route or proportion (through the caveolin-mediated endocytosis and macrophage pathway), thereby reducing the disruption and degradation of siRNA in the cell, to further improve the silencing activity of siRNA, and finally achieve the high efficiency and safety of siRNA delivery.

Isonucleosides are nucleoside analogs in which the position of a nucleobase is shifted from 1′ to the 2′ position of a glycosyl group. Stability of the glucosidic bond will increase by this shift of base. Our laboratory has synthesized two kinds of isonucleosides of D-(formula I) and L-(formula II) of different configurations using different raw materials. The general formula of the isonucleosides are is set forth as follows:

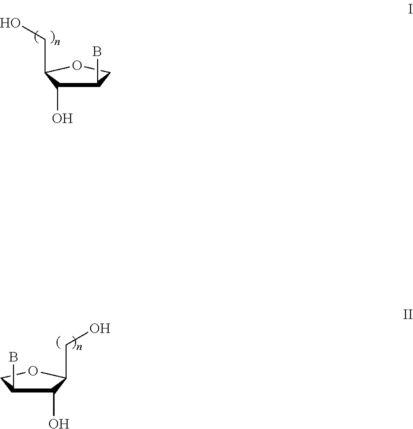

Wherein n=1, 2, 3; B is thymine (T), uracil (U), cytosine (C), guanine (G) or adenine (A). Among above chemical formulae, isonucleoside (formula I) is D-configuration, and (formula II) is L-configuration.

In the previous work of the present invention, it has been demonstrated that different configurations of D-/L-isonucleosides can have different effects on the local conformation of the oligonucleotides, thereby affecting their physicochemical properties and silencing activity. In addition, since D-/L-isonucleosides are structurally highly similar to native nucleosides, the original properties of the oligonucleotides can be retained or approximated to the maximum extent possible and can be applied as a pair of molecular probes applied in the areas, such as exploration of the interaction mechanisms between nucleic acids and proteins.

In the present invention, the peptide conjugation is preferably modified to the 3′ end of the sense- and/or antisense-strands of siRNA, the peptide fragment conjugated may be a peptide mimetic. The general formula of linkage between peptide conjugated fragments and RNA part is as follows:

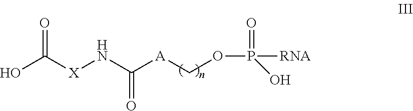

Wherein X is a polypeptide sequence, A is a substituted or unsubstituted phenyl ring structure or a carbon atom, n is 0, 1, 2, 3, 4, or 5.

Cell permeable peptides can mediate variety of molecules cross the cell membrane, the use of cell permeable peptide-mediated DNA and PNA intracellular transport has been widely used and achieved good results. Starting from the Kaposi FGF signal peptide (15 peptide), in a early stage study in our laboratory, a 6 peptide sequence H-Leu-Ala-Leu-Leu-Ala-Lys-OH (KALLAL) with hydrophobic characteristics was obtained by computer simulation. The result of covalent conjugation of PNA and antisense nucleic acid, respectively, with the above sequence showed that it can not only improve the stability and silencing activity of these two nucleotides, but also improve the transmembrane capacity of the modified oligonucleotide. In particular embodiments of this invention, the polypeptide sequence represented by X in Formula III is a KALLAL or a similar peptide sequence thereof.

Gemini type cationic liposome vectors usually consist of cationic heads, aliphatic tails and a tether. Structures of the head are cationic groups such as peptides, glycolipids and so on, the tails are usually saturated or unsaturated long aliphatic chains, cholesterol and other hydrophobic molecules, the tether is mostly disulfide linkages or amide linkages which can be degradable in vivo. This kind of vector can effectively compress and aggregate siRNA on the surface of lipid particles of vectors to form nanocomposites through the electrostatic adsorption between the negatively charged cationic water-soluble heads and the phosphoric acid skeleton of gene drug (DNA/PNA/siRNA). When this complex is taken up by the cells, the composite structure changes from lamellar to hexahedral shaped due to changes in the environment inside and outside the cell membrane, causing the composite to be disassembled and to escape from the lysosome to the cytoplasm and release DNA/RNA, which in turn generates silent effect.

In the present invention, preferably the cationic liposome vector may be a commercial one, such as RNAiMax, Lipofectamine and the like, or a Gemini type cationic liposome vector of the general Formula IV:

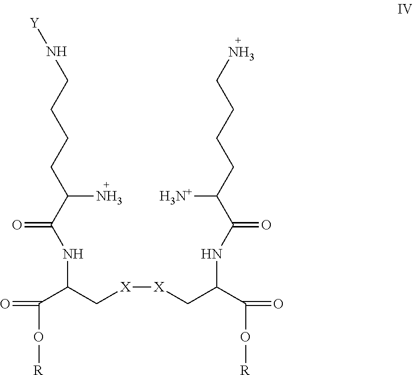

Wherein X is a sulfur atom (S) or a carbon atom (C), Y is a positively charged nitrogen-containing group or targeting group and R is a saturated or unsaturated aliphatic chain or a hydrophobic molecule.

In a specific embodiment of the present invention, the unsaturated aliphatic chain represented by R in Chemical Formula IV is oleylgroup.

In the present invention, preferably the incorporation of a D- or an L-isonucleosides at one or more sites of the sense and/or antisense strands of siRNA is achieved by solid-phase synthesis That is to say, using an isonucleosidephosphoramidite monomer in place of the natural nucleoside phosphoramidite monomer to carry out conjugation at the corresponding incorporation position.

Before carrying out siRNA modification, the isonucleoside compounds of Formula I and/or Formula II are respectively prepared into isonucleosidephosphoramidite monomers of formula V and/or formula VI. In each cycle a nucleoside is conjugated, each cycle includes four reactions: DMT de-coupling, conjugating, blocking, oxidation.

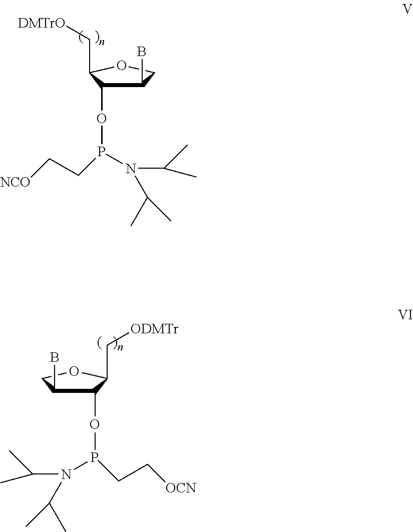

Wherein n=1, 2, 3; B is thymine (T), uracil (U), amino-protected guanosyl (G), adenyl (A) or cytosine (C).

In the present invention, preferably the conditions for synthesizing the DNA oligonucleotide chain are such that the number of injections of the isonucleosidephosphoryl monomer is increased up to three times; the coupling time after each injection is 300 seconds/each time. The conditions for the synthesis of isonucleoside-modified RNA oligonucleotide strands are such that the coupling time after each cycle of injection is increased to 900 seconds/each time, coupling three times.

In the present invention, preferably said integrated chemical modification method may further includes the joint use of common chemical modification strategies including 2′-O-methoxy, 2′-fluoro (2′-F), locked nucleotides (LNA), phosphosulfur skeleton modification and other terminal conjugation methods.

In a particular embodiment of this invention the siRNA sequence to be modified is a siMek1 sequence that targets the mRNA for the MEK1 protein in the ERK pathway as well as a siMB3 sequence that targets the mRNA for the variant B-Raf kinase protein in the ERK pathway. Said sequences siMek1 and siMB3 before modifications are as follows:

siMek1:

| | |
|---|---|
| sense strand: | 5′-GCAACUCAUGGUUCAUGCUdtdt-3′; |
| anti sense strand: | 5′-AGCAUGAACCAUGAGUUGCdtdt-3′ |

siMB3:

| | |
|---|---|
| sense strand: | 5′-GCUACAGAGAAAUCUCGAUdtdt-3′ |
| antisense strand: | 5′-AUCGAGAUUUCUCUGUAGCdtdt-3′ |

In a specific embodiment of the present invention, when the following two conjugates: the peptide fragment of Formula III conjugated to the 3′ end of the sense as well as antisense strands of the siMB3 sequence described above (PA/PS-siMB3), and, the peptide fragment of Formula III conjugated to the 3′ end of only the sense strand of the siMB3 sequence (PS-siMB3), were compared with the peptide fragment of Formula III (PA-siMB3) conjugated to the 3′ end of the antisense strand, as well as the unmodified siMB3 sequence, the PA/PS-siMB3 and PS-siMB3 sequences showed the higher serum stability.

In a specific embodiment of the present invention, the first nucleotide at the 5′ end of the sense strand of the siMek1 as well as the siMB3 sequence described above were incorporated with the isonucleoside represented by Formula I or Formula II, and at the 3′ end of the sense and antisense strands were conjugated with the peptide fragment of Formula III, and was encapsulated by the cationic vector RNAiMax, or the cationic liposome vector Lipofectamine, or with cationic liposome formed as Formula IV. In comparison with the unmodified siMek1 and siMB3 sequences encapsulated with cationic liposome, the above-mentioned modified sequences demonstrated higher silencing activity.

In a specific embodiment of the invention, the peptide fragment of Formula III was conjugated to the 3′ end of the sense and antisense strands of the siMB3 sequence described above, and the cationic liposome material formed as Formula IV was used as a delivery vector, after explorations and investigations on the preparation process conditions and other related parameters, the preparation method of two different siRNA/vector composites were ultimately determined studies showed that the resulting siRNA/vector composite showed lower cytotoxicity and higher stability.

In a specific embodiment of the present invention, when the peptide fragment of Formula III is conjugated to the 3′ end of the sense and antisense strands of siMek1—as well as siMB3 sequences as described above, and is encapsulated with the cationic liposome formed according to Formula IV, could result in homogeneous and stable distribution of specific spherical vesicles. Compared with the unmodified siMek1 and siMB3 sequences encapsulated with cationic liposomes formed according to Formula IV, both of the encapsulated, modifies iMek1- and siMB3 sequences resulted in stronger intermolecular interactions, as well as formation of composites with a lower surface potential, larger particle size and denser internal nano-assembly structure.

In a specific embodiment of the invention, the peptide fragment of Formula III was conjugated to the 3′ end of the sense and antisense strands of the siMB3 sequence described above, and encapsulated with cationic liposomes of Formula IV to form nanocomposite, which could enter into cells (the caveolin-mediated endocytosis and macrophage pathway) in the desired pathway or proportion, thereby bypassing to some extent the intracellular lysosomal degradation process, realizing exertion of high efficient of the silence activity.

In a specific embodiment of the invention, the peptide fragment of Formula III was conjugated to the 3′ end of the sense and antisense strands of the siMB3 sequence described above, the formed nanocomposite was encapsulated with cationic liposomes formed according to Formula IV. Compared with unmodified siMB3 sequence encapsulated with cationic liposomes, that produced with modifies iMB3 sequence showed a higher silencing activity.

This invention realizes the controllability of the assembled composites through the optimization of the conditions in formulation process in which the 3′,3″-bispeptide-siRNA conjugates were encapsulated with a cationic liposome vector (the general Formula IV), optimizations also including the optimization of parameters such as particle morphology, internal structure, particle size, electric potential and so on.

Most preferably, said integrated chemical modification method includes the following steps:

(1) Weigh out CLD solid powder, dissolve in anhydrous ethanol to obtain an ethanol solution of CLD, preserve at 4° C.;

(2) Modify 3′-end of sense and antisense strands of siRNA with peptide conjugation, and hydrate it by using the DEPC water;

(3) Add the CLD ethanol solution obtained in steps (1) and the siRNA after DEPC water hydration in steps (2) according to ratios of V_{siRNA}/V_{CLD}=5/1, N/P=3/1 or 5/1, sonicate for 40 min under a temperature of 70° C., subsequently vortex for 1 min to obtain nano-composite particles.

The route or proportion of the formed nanocomposite particles entering into the cells can be regulated through caveolin-mediated endocytosis and macropinocytosis.

Compared with the existing technique, embodiments of the present invention have at least the following advantages:

1. The chemical modification strategy provided by this invention combines the modification of siRNA with isonucleosides, with terminal peptide and with encapsulating of cationic liposome vector, thereby bringing into play the respective advantages of the three chemical modification methods used and avoiding their disadvantages, the obtained siRNA has higher serum stability and biological activity, and showed good and controllable transmembrane transport ability and silencing effect to the target mRNA, laying a good foundation for the clinical application of siRNA technology.

2. There are dual binding effects between 3′,3″-bispeptide-siRNA conjugate and cationic liposome vector, when a smaller amount of vector dosage is used, biological activity similar to the commercial vector can be achieved, realizing high siRNA delivery efficiency and reducing the biological toxicity caused by the vector itself.

3. Through a comprehensive study on results of serum stability and biological activity after modification of D-, L-isonucleoside on different sites of siRNA, differences of impacts due to conformational changes of different sites of siRNA could be obtained.

4. Through the exploration and optimization of preparation conditions and parameters for the formation of nanocomposites with siRNA and cationic liposome vector, a homogeneous and stable assembly system can be obtained and its regulation of the transmembrane pathways can be achieved, thereby affecting its intracellular metabolism behavior, ultimately play highly active silencing role, which will further promote the clinical application of siRNA.

Embodiments utilize a joint modification with isonucleoside modification as well as terminal peptide conjugation and cationic liposome vector encapsulation, the product obtained by this chemical modification method has the characteristic of stable physical and chemical properties, well controllable biological behaviors, and highly efficient bio-activities, they can be widely used in anti-virus, and anti-cancer drug researches.

### BRIEF DESCRIPTION OF THE ATTACHED DRAWINGS

FIG. 1 shows the results of serum stability (50% FBS) of isonucleoside conjugated 3′,3″-bispeptide-siMB3 conjugate.

FIG. 2 shows the results of the silencing activity (30 nM, 24 h) of isonucleoside conjugating 3′,3″-dipeptidyl-siMek1 conjugate. The transfection reagent is cationic liposome RNAiMax. The upper panel shows the results of Western Blotting, The FIG. below shows the result of real-time PCR.

FIG. 3 shows the results of real-time PCR (30 nM, 24 h) of isonucleoside conjugated 3′,3″-bispeptide-siMB3 conjugate. The transfection reagent is cationic liposome RNAiMax.

FIG. 4 shows the results of real-time PCR (30 nM) of isonucleoside conjugating 3′,3″-bispeptide-siMB3 conjugate. The transfection reagent is cationic liposome Lipofectamine.

FIG. 5 shows the results of Western blotting (30 nM) of isonucleoside conjugating 3′,3″-bispeptide-siMB3 conjugate. The transfection reagent is cationic liposome Lipofectamine.

FIG. 6 shows the result of the stability of the cationic liposome vector/3′,3″-bispeptide-siRNA conjugates detected by agarose gel retardation electrophoresis (where siMek1** indicates the 3′, 3″-bispeptide siMek1 conjugate). The samples were prepared by mixing SiMek1 at N/P=1, 2, 4, 6, 8, 10 and 12, respectively, with the cationic liposome vector and incubating at room temperature for 30 min, and then separating with electrophoresis at 110 V for 30 min.

FIG. 7 shows the effect of the parameters in two-phase method (MT method) on the formation of native and 3′,3″-bispeptide-siRNA conjugates and cationic liposome vector CLD preparations.

FIG. 8 is a preparation formed by combining the optimal conditions in the MT preparation process.

FIG. 9 is an in vitro qualitative evaluation (serum stability test, erythrocyte hemolysis test, and dilution stability test) of four formulations formed with native as well as 3′,3″-bispeptide-siRNA encapsulated with cationic liposome vector CLD.

FIG. 10 is a dynamic light scan (DLS) results of four formulations of native as well as 3′,3″-bispeptide-siRNA encapsulated with the cationic liposome vector CLD.

FIG. 11 is a transmission electron microscopy (TEM) image of a cationic liposome vector encapsulating a native siRNA and a 3′,3″-bispeptide-siRNA conjugate to form formulations (AT method). Wherein A is morphology diagram of the cationic liposome vector CLD; B is an ionic pattern of a cationic liposome vector CLD and a native siRNA; C is morphology diagram of a cationic liposome vector CLD bound to a 3′,3″-bispeptide-siRNA conjugate (N/P=20, m_{siRNA}=200 ng). Bar: 200 nm; Bar in square: 100 nm.

FIG. 12 is an atomic force microscopy (AFM) diagram of the formulations formed by cationic liposome vector CLD encapsulating native siRNA and 3′,3″-bispeptide-siRNA conjugate (AT method). Wherein A is morphology diagram of cationic liposome vector CLD per se; B is that of cationic liposome vector CLD encapsulating natural siRNA; C is that of cationic liposome vector CLD encapsulating 3′,3″-bispeptide-siRNA conjugate (N/P=20, m_{siRNA}=200 ng).

FIG. 13 is the cellular uptake of four formulations formed by binding of native siRNA as well as 3′,3″-bispeptide-siRNA conjugates with cationic liposome vector CLD.

FIG. 14 shows transmembrane pathways and the choice of small molecule cell pathway inhibitors of each channel.

FIG. 15 shows the cellular uptake after treatment with inhibitors of the four formulations formed by binding of native siRNA as well as 3′,3″-bispeptide-siRNA conjugates with cationic liposome vector CLD.

FIG. 16 is a diagram of the ratios of different endocytic pathways of four formulations formed by binding of the cationic liposome vector CLD with native siRNA as well as 3′,3″-bispeptide-siRNA conjugates.

FIG. 17 shows the RT-PCR results of silencing activities of the four formulations formed by binding of cationic liposome vector CLD with native siRNA as well as 3′,3″-bispeptide-siRNA conjugates at different times and different concentrations.

FIG. 18 shows the results of the silencing activity of natural and isonucleoside conjugating 3′,3″-bispeptide-siRNA conjugates transfected by the cationic liposome vector CLD. Wherein Blank is a natural siRNA, Na is a cationic liposome vector CLD transfecting natural siRNA, PP is a cationic liposome vector CLD transfecting 3′,3″-bispeptide-siRNA conjugate, D1PP is a cationic liposome vector CLD transfecting d-isosucleoside modified 3′,3″-bispeptide-siRNA conjugate, L1PP is a cationic lipids vector CLD transfected L-isonucleoside modified 3′,3″-Bispeptide-siRNA conjugate.

FIG. 19 shows the stability of the formulations of nanocomposites formed by joint modification of the cationic liposome vector CLD and isonucleoside to 3′,3″-bispeptide-siRNA conjugates.

### DETAILED DESCRIPTION

The present invention will be further described with reference to specific embodiments. The advantages and features of the present invention will be more apparent from the description of specific embodiments. However, these embodiments are merely exemplary and do not limit the scope of the present invention in any way. It should be understood by those skilled in the art of this field that the technical solutions and details of the present invention may be modified or replaced without departing from the spirit and scope of the present invention, and all such modifications and substitutions fall into the protection scope of the present invention.

### Embodiment 1. Synthesis of siRNAs Co-Modified by Isonucleoside and Terminal Conjugation with Peptide, and Evaluation of Serum Stability of the Synthetic Product

1. Synthesis of siRNA Co-Modified by Isonucleoside and Terminal Conjugation with Peptide

In this embodiment, the siRNA to be modified is a siMB3 sequence that targets mRNA of a variant BRaf kinase protein in the ERK pathway. The pre-modified siMB3 sequence is as follows:

| siMB3: | |
|---|---|
| sense strand: | 5′-GCUACAGAGAAAUCUCGAUdtdt-3′ |
| antisense strand: | 5′-AUCGAGAUUUCUCUGUAGCdtdt-3′ |

The modification strategy selects one of the following: 1) The first nucleotide of the 5′ end of the sense strand of the siMB3 sequence as described above is conjugated with the isonucleoside of Formula I or Formula II.

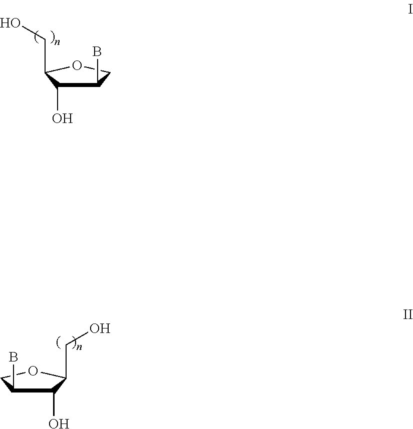

Wherein n=1; B is guanosyl (G).

2) Conjugating the peptide fragment of Formula III to the 3′ terminus of the sense and antisense strand of said siMB3 sequence (PA/PS-siMB3), or, only to the 3′ end of sense- or antisense strand of said siMB3 sequence (PS-siMB3/PA-siMB3).

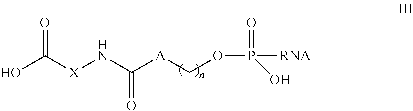

Wherein X is a 6-peptide sequence H-Leu-Ala-Leu-Leu-Ala-Lys-OH (KALLAL), A is a carbon atom and n is 1.

3) Conjugating the peptide fragment of Formula III to the 3′ end of the sense and antisense strand (PA/PS-siMB3) or, only to the 3′ terminus of the sense or antisense strands of said siMB3 sequence described above (PS-siMB3/PA-siMB3), and the 5′ end of first nucleotide of sense strand of the siMB3 sequence described above is conjugated with the isonucleoside of Formula I or Formula II.

The modified siRNA obtained applying the above modification strategy is shown in Table 1 below:

TABLE 1

| Name of siRNA | Description |
|---|---|
| siMB3-S01D | The first nucleotide of 5′ end of the sense strand of the siMB3 |
| | sequence is conjugated with the isonucleoside of Formula I |
| siMB3-S01L | The first nucleotide of 5′ end of the sense strand of the siMB3 |
| | sequence is conjugated with the isonucleoside of Formula II |
| siMB3-PepS | Peptide fragment of Formula III is conjugated to the 3′ end of the |
| (PS-siMB3) | sense strand of the siMB3 sequence |
| siMB3-PepAs | Peptide fragment of Formula III is conjugated to the 3′ end of the |
| (PA-siMB3) | antisense strand of the siMB3 sequence |
| siMB3-PepS/PepAs | The peptide fragment of Formula III is conjugated to the 3′ |
| (PA/PS-siMB3) | terminus of the sense and antisense strands of the siMB3 sequence |
| siMB3-S01D-PepS | While the 3′ terminus of the sense strand of the siMB3 sequence is |
| (PS-siMB3-S01D) | conjugated with the peptide fragment of Formula III, the first |
| | nucleotide at the 5′ end of the sense strand of the siMB3 sequence |
| | is conjugated with the isonucleoside of Formula I |
| siMB3-S01L-PepS | While the 3′ terminus of the sense strand of the siMB3 sequence is |
| (PS-siMB3-S01L) | conjugated with the peptide fragment of Formula III, the first |
| | nucleotide at the 5′ end of the sense strand of the siMB3 sequence |
| | is conjugated with the isonucleoside of Formula II |
| siMB3-S01D-PepAs | While the 3′ end of the antisense strand of the siMB3 sequence is |
| (PA-siMB3-S01D) | conjugated with the peptide fragment of Formula III, the first |
| | nucleotide at the 5′ end of the sense strand of the siMB3 sequence |
| | is conjugated with the isonucleoside of Formula II |
| siMB3-S01L-PepAs | While the 3′ end of the antisense strand of the siMB3 sequence is |
| (PA-siMB3-S01L) | conjugated with the peptide fragment of Formula III, the first |
| | nucleotide at the 5′ end of the sense strand of the siMB3 sequence |
| | is conjugated with the isonucleoside of Formula II |
| siMB3-S01D-PepS/PepAs | While the peptide fragment of Formula III is conjugated to the 3′ |
| (PA/PS-siMB3-S01D) | terminus of the sense and antisense strands of the siMB3 sequence, |
| | the first nucleotide of the 5′ end of the sense strand of the siMB3 |
| | sequence is conjugated with the isonucleoside of Formula II |
| siMB3-S01L-PepS/PepAs | While the peptide fragment of Formula III is conjugated to the 3′ |
| (PA/PS-siMB3-S01L) | terminus of the sense and antisense strands of the siMB3 sequence, |
| | the first nucleotide of the 5′ end of the sense strand of the siMB3 |
| | sequence is conjugated with the isonucleoside of Formula II |

2. Experiment on Serum Stability

The stability of the modified siMB3 sequence in 10% (volume fraction) fetal bovine serum was investigated respectively. 4 μL (20 μM) of natural or differently modified siMB3 sequence+20 μL of FBS+16 μL PBS were added into 200 μL microcentrifuge tubes, respectively, mixed, and μL of the mixtures were transferred to 3 microcentrifuge tubes, respectively, incubated in 37° C. water bath, the samples were simultaneously transferred immediately into a −80° C. refrigerator or liquid nitrogen. After adjusting of the temperature on ice, the mixtures were analyzed by using 20% denatured polyacrylamide gel electrophoresis, stained with nucleic acid dye for 15 min, finally, the chemiluminescence gel imaging system was used for imaging analysis.

3. Results

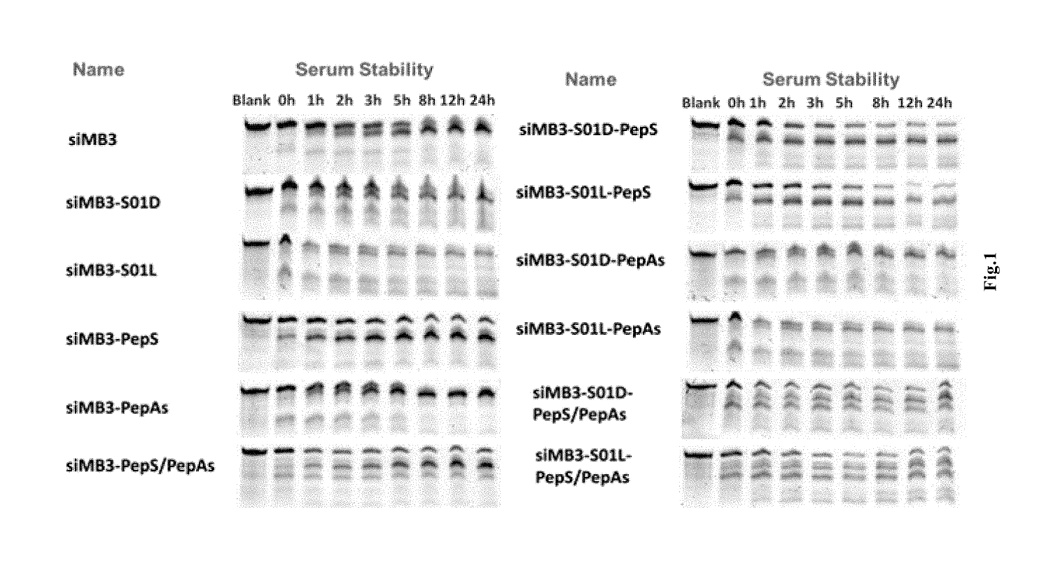

The experimental results are shown in FIG. 1. From the results it can be seen that when only at the 5′ end of the sense strand was co-modified with D-isonucleoside and peptide conjugation, the effect of D-isonucleoside on serum stability was insignificant, whereas that with L-isonucleoside reduced serum stability to a certain extent; however, after conjugation with monopeptide, the terminus conjugation on sense and antisense showed different results, the sense strand monopeptide- and bispeptide conjugation showed consistant results and they all significantly improved serum stability. However, the antisense strand modifications showed less impact on serum stability. When isonucleoside modifications were made at the 5′ end of the sense strand, regardless of D- or L-isonucleoside modifications the monopeptide conjugation modified on the antisense strand would increase the serum stability, whereas sense strand monopeptide conjugation would lead to decrease of serum stability. This suggested that the end-group selectivity of ribozymes was related to thermodynamics, whereas isonucleoside would alter the balance. Peptide conjugation modifications compensated for the effect of different isonucleoside modifications on serum stability and allowed for modification strategies that resulted in a substantial increase in stability.

### Embodiment 2. Biological Activity Evaluation on siRNA/Cationic Liposomes RNAiMax Co-Modified with Isonucleoside and Terminal Peptide Conjugate

Experimental operation: 100,000 Hela cells/well were added into 6-well plate, cultured overnight. 5 μL of RNAiMAX was added with modified siRNA at a final concentration of 30 nM, incubated at room temperature for 15 min, the samples were add into the cell culture plate and incubated for 24 h. The total RNA was extracted using TRizol, the total RNA were reverse transcribed into cDNA. Real-time experiments performed with Gotaq Green Mix to investigate the gene silencing effect of siRNA on the mRNA level. Total protein was extracted, quantified using BSA as benchmark, and separated by means of polyacrylamide gel (separation gel 10%, concentrated gel 5%), subsequently, after transferring the proteins from the gel, primary and secondary antibodyimmunal reaction were carried through. Protein knockout effect was detected using Biorad chemiluminescence gel detection system.

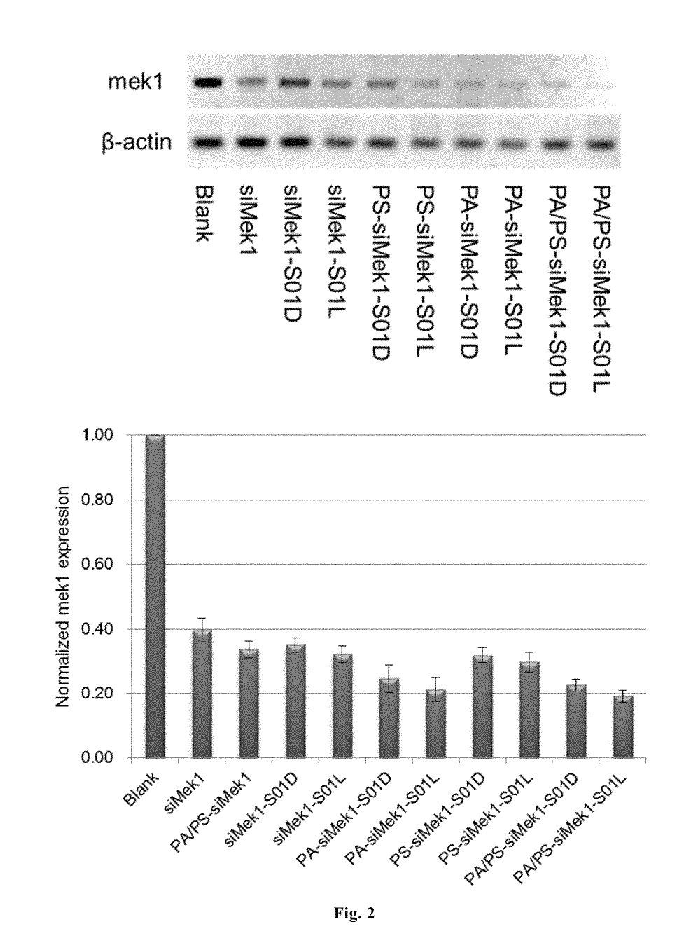

Experimental Results: According to the PCR as well as real-time PCR results (FIG. 2), when the siMek1 sequence targeting the mRNA of the MEK1 protein in the ERK pathway was modified, the isonucleoside modification is performed at the 5′ end of the sense strand, whether without peptide conjugation or with different peptide conjugation modifications, it was found that the improvement of silencing activity of siRNA with D-isonucleoside modification was more obvious in comparison with that with L-isonucleoside. Comparisons with the conjugation of different peptides showed that the order of the increase of activities after modifications was: with monopeptide to sense strand< with monopeptide to antisense strand< with bispeptide to antisenses strand. Besides, regardless of D- or L-isonucleoside modification to the sense strain 5′ terminus, the results showed the same regularity of change in activity. The same change in activity was obtained when only peptide conjugation was done without modification with isonucleosides, indicating that the effect of isonucleoside modification and peptide conjugation modification on the activity are additive to each other. Therefore, the most obvious modification strategy of increasing activity was found by PCR- and real-time PCR results: 3′-3″-bispeptide-siRNA conjugate modified with L-isonucleoside on the 5′ end of the sense strand.

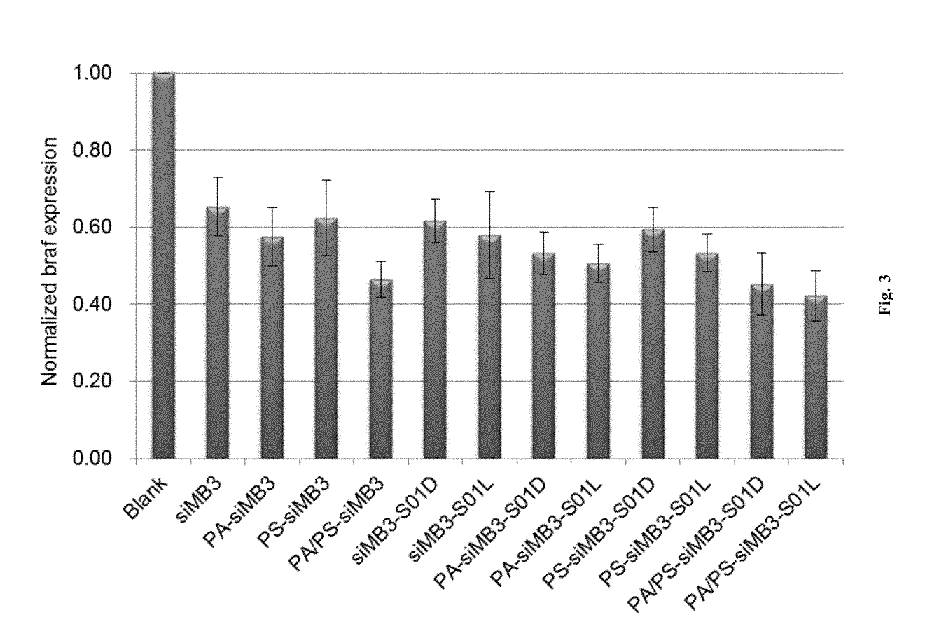

When performing the modification and activity evaluation of siMB3 targeting Braf-mutant mRNA, in all the siMB3 leading structures, it was found that according to the results of real-time PCR (FIG. 3), modification with L-isonucleoside on 5′-end of the sense strain combined with 3′,3″-bispeptide-siRNA conjugate led to the best silencing activity. However, it was different from the modified siMek1 sequence that the silencing activity of 3′,3″-bispeptide-siRNA conjugate was found to be significantly higher in the siMB3 modification, even higher than the result obtained from modification of siRNA with L-isonucleoside conjugating to 5′-end sense strain. At the same time, the effects of D-/L-isonucleoside modifications on silencing activity were also not evident as in siMek1. By comparing the serum stability of siMek1 and siMB3 made before, that from siMek1 was found to be significantly more stable in serum than that from siMB3. Because of the lower serum stability of siMB3, the enhancement of serum stability by the bispeptide conjugation in siMB3 was also more pronounced. The increased serum stability of siRNA meant that there were more intact siRNAs present in the body and therefore more siRNAs would exist that would exert their silencing activity. Therefore, because siMB3 showed more pronounced enhancement of the stability of the silencing activity, for siMB3, with bispeptide conjugation the increase in serum stability was also more pronounced, whereas the effect of D-/L-isonucleosideswas insignificant, indicating that as far as the increase of silencing activity is concerned, modification of 5′ end of the sense strand showed sequence specificity.

This might be related to the existence of different degrees of competition between entrance of different sequences of sense and antisense double strands into the RISC complex. It is preliminarily speculated that the antisense strand would have a significant edge when the thermodynamic difference between the 5′ end of the sense- and the antisense-strand was large (siMB3), so the effect of sense strand restricted to enter RISC by the isoNA modification at the 5′ end of the sense strand would not be significant. During formation of the RISC composite, the ability of the sense- and antisense strands to enter the RISC complex is close to each other, therefore, when isonucleoside modification on the 5′ end of sense strand blocks the ability of the sense strand to enter the RISC complex, the ability of the antisense strand to enter the RISC complex will increase much more, thereby increasing the silencing activity more profound.

### Embodiment 3. Evaluation of Biological Activity of 3′, 3″-Bipeptide-siRNA Conjugate/Cationic Liposome Lipofectamine with Joint Isonucleoside Modification

Experimental Procedures: A375 cells was added into 6-well plate at 100,000 cells/well and cultured overnight. 4 μL Lipofectamine was mixed with modified siRNA at a final concentration of 30 nM, incubated at room temperature for 20 min, added to cell culture plate and incubated for 24 h-72 h. The total RNA was extracted by TRizol, and was reverse transcribed it into cDNA. Real-time experiments were performed with Gotaq Green Mix to investigate the gene silencing effect of siRNA at the mRNA level. The total protein was extracted and was quantified using BSA as benchmark. The proteins were separated by polyacrylamide gel (separation gel 10%, concentrated gel 5%), and then after transferring the proteins from the membrane, primary and secondary antibodyimmunal reactions were carried through. Protein knockout effect was detected by using Biorad chemiluminescence gel detection system.

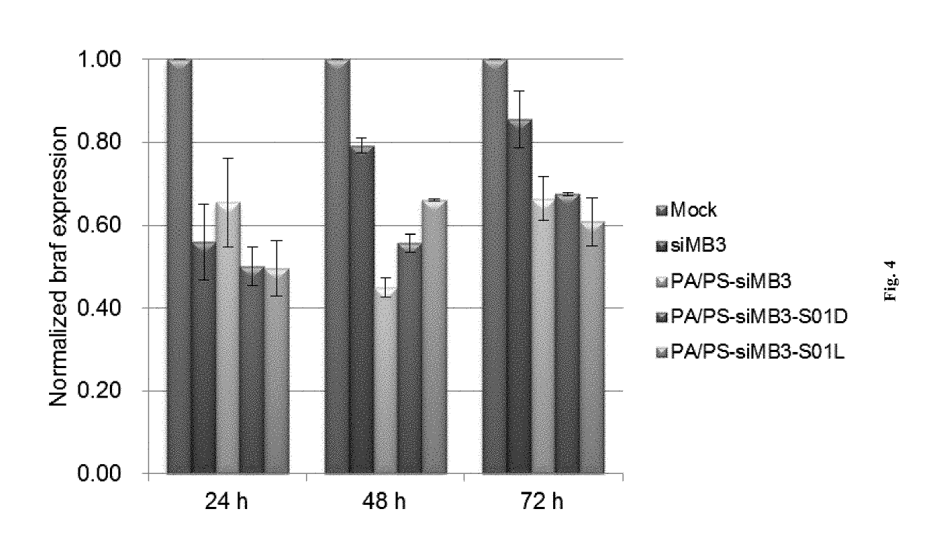

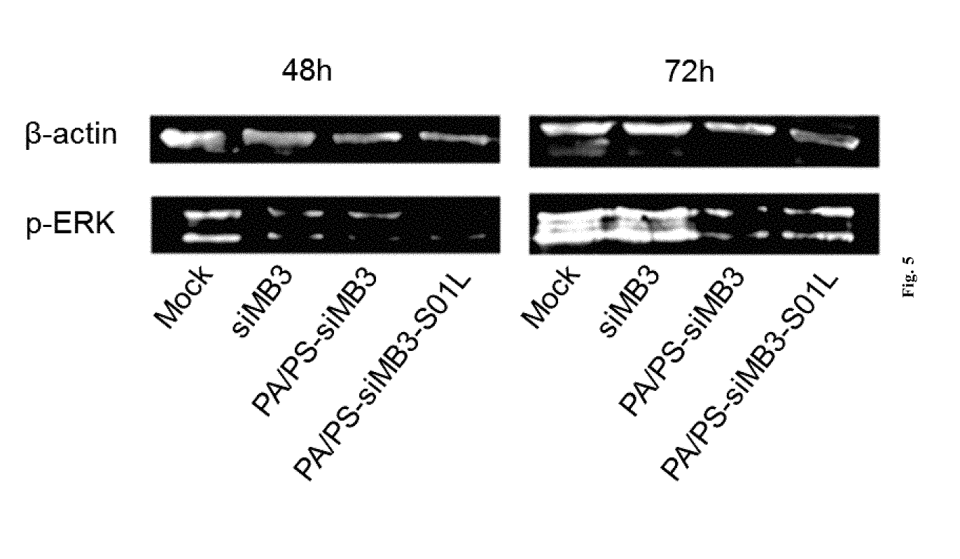

Results: Real-time PCR result of the 30 nM leading structures of siRNA transfected by Lipofectamine (FIG. 4) showed that after incubating for 24 h, the silencing activities of leading structures PA/PS-siMB3-S01D and PA/PS-siMB3-S01L were both slightly better than natural siMB and 3′,3″-bispeptide-siRNA conjugates (PA/PS-siMB3), whereas PA/PS-siMB3 had the weakest silencing activity among them. Silencing activity of native siMB3 was significantly reduced when the incubation time was extended to 48 h, while although the silencing activity of leading structures PA/PS-siMB3-S01D and PA/PS-siMB3-S01L slightly decreased, their silencing activities were still better than natural siMB3. However, the silencing activity of 3′,3″-bispeptide-siRNA conjugates were significantly increased compared with those after 24 h incubation. When the incubation time extended to 72 h, mRNA levels decreased from the beginning due to degradation of mRNA by siRNA began to be restored. The reason could be owing to continuous transcription during extension of incubation time. However, the comparison of different modified sequences revealed that the bispeptide conjugation, and the bispeptide conjugation combined with the modification of the 5′-end of the sense strand with L-isonucleoside modification had the best silencing activity, indicating that the bispeptide conjugation could to some extent prolong the time for the siRNA to implement its silencing activity. Preliminary analysis showed that the prolongation of silencing effect should relate significantly to the increase of the intracellular stability of the siRNA. Moreover, in combination with the regularity of biodegradation in the serum of the bispeptide conjugated siRNA, it could be found that the conjugated peptide mostly preferentially degraded prior the degradation inside the double strand of siRNA, that is, the peptide conjugation could hinder the attack of the ribozyme on the end of the siRNA. When the conjugated peptide had not been cut down, it could delay the attack on siRNA by ribozyme. On the other hand, it may also delay the recognition of Dicer- and TRBP protein and thus play a role in delaying exertion of the silencing activity of siRNA. Therefore, the 3′,3″-bispeptide-siRNA conjugate sequence has better silencing activity than the native siRNA when the incubation time was extended to 72 h. According to the results of Western blotting (FIG. 5), after an incubation time of 48 h, the silencing activity of the leading structure PA/PS-siMB3-S01L was also significantly better than that of native siMB3- and 3′,3″-bispeptide-siRNA conjugate, according to the expression of the downstream protein p-ERK. Silencing activity of 3′,3″-bispeptide-siRNA conjugate and leading structures PA/PS-siMB3-S01L were still better than that of native siMB3 when the incubation time was extended to 72 h.

### Embodiment 4. Examination of the Binding Capability of 3′,3″-Bispeptide-siRNA Conjugates to the Cationic Liposome Vector CLD Using Agarose Gel Electrophoresis

Preparation of 3′,3″-bispeptide-siRNA Conjugates:

The pre-modification siMek1 sequence was as follows:

siMek1:

| | |
|---|---|
| sense strand: | 5′-GCAACUCAUGGUUCAUGCUdtdt-3′; |
| antisense strand: | 5′-AGCAUGAACCAUGAGUUGCdtdt-3′ |

Peptide fragment of Formula III (PA/PS-siMek1) is conjugated to the 3′ terminus of both the sense and antisense strands of the siMek1 sequence:

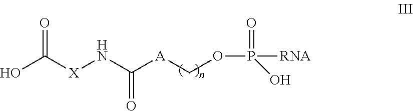

Wherein X is a 6-peptide sequence H-Leu-Ala-Leu-Leu-Ala-Lys-OH (KALLAL), A is a carbon atom and n is 1.

The specific structure of the CLD molecule is as follows: Lysine is used as the cationic head, the tether is consists of cystine residues, and oleyl (an octadecenyl having a 9,10-cis double bond) constitutes the lipophilic tails.

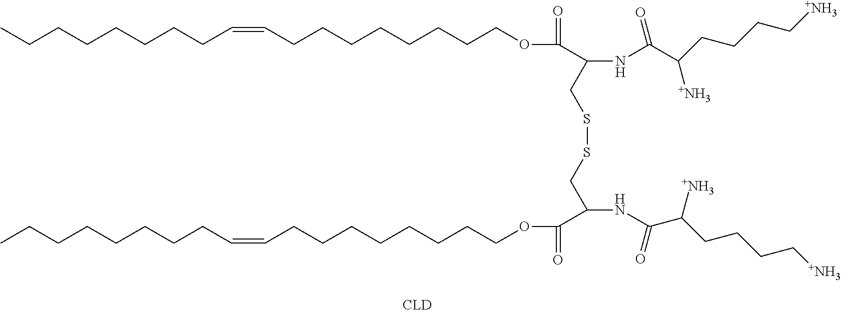 [SMILES: CCCCCCCC/C=C\CCCCCCCCOC(=O)C(CSSCC(NC(=O)C([NH3+])CCCC[NH3+])C(=O)OCCCCCCCC/C=C\CCCCCCCC)NC(=O)C([NH3+])CCCC[NH3+]]

Experimental procedures (AT method): 4.2 mg CLD was weighed out into a silanized Erlenmeyer flask, dissolved in a certain amount of organic solvent (chloroform/methanol=1:1, v/v). A flow of nitrogen or argon was used to dry the solvent, making the solution into an adherent film. The residual solvent was vaporized slowly under reduced pressure. The film was removed from wall of flask, placed in a vacuum dryer and dried overnight. On the next day, the dried film was removed, adding 1 mL of DEPC water, the container was plugged with a stopper. The reaction mixture as sonicated for 30 min in 50° C. water bath, filtered with 0.25 micron sterile membrane to prepare the cationic liposome. The just prepared cationic liposome composite (4.2/mL, corresponding to the concentration of CLD at N/P=20) was added with siRNA aqueous solution containing 20 μM siRNA, and Opti as solvent, solutions equivalent to N/P=1, 2, 4, 6, 8, 10 and 12 transfection reagent were prepared, after adding 3′,3″-bispeptide-siRNA conjugate water solution, the solution was incubated at room temperature for 30 min to complete the preparation of CLD/3′,3″-bispeptide-siRNA conjugate composite. The binding ability of 3′,3″-bispeptide-siRNA conjugates to CLD was investigated by agarose gel electrophoresis retardation assay under the following experimental conditions: 1% agarose gel; 1×TAE electrolytic solution; Voltage 110 V; electrophoretic time 30 min; use Goldview as dye; the cationic vector is CLD.

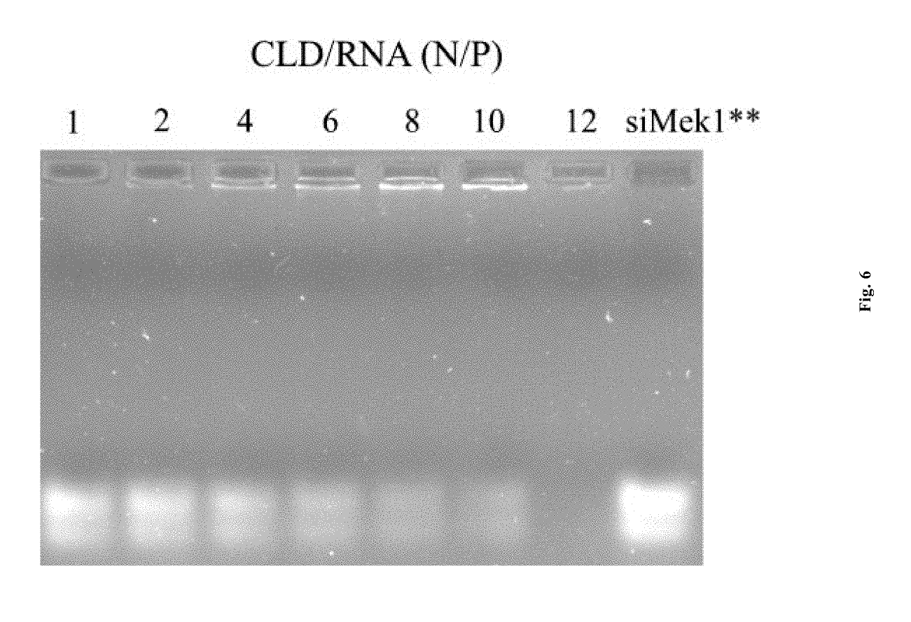

Experimental Results: It can be seen from FIG. 6 that as the N/P increases, the cationic liposome vector (Formula IV, R=oleyl alcohol) could be effectively conjugated to 3′,3″-bispeptide-siRNA, and eventually be able to complete the reaction. When N/P=1, the cationic liposome vector already begins to be able to effectively bind to the 3′,3″-bispeptide-siRNA conjugate. As N/P increases, the brightness of the band of free 3′,3″-bispeptide-siRNA conjugate further diminished. At N/P=12, the brightness of the free bis 3′, 3″-bispeptide-siRNA conjugate band was very vague relative to the brightness of control siRNA band, at which point the cationic liposome vector compound binds completely to the 3′,3″-bispeptide-siRNA conjugate (FIG. 6). The results of binding capability of siRNA with 3′,3″-bispeptide-siRNA conjugates again demonstrated that the presence of dual effects enhanced the capability of siRNA binding to the cationic liposome vector.

### Embodiment 5 Exploration and Determination of Two-Phase Mixing Method (MT Method) Process Parameters for the Preparation of Nanocomposite with Natural as Well as 3′,3″-Bispeptide-siRNA Conjugates Binding to Cationic Liposome Vector CLD

Setting of conditional parameter: a certain concentration of CLD ethanol solution was slowly added dropwise to a certain concentration of siRNA aqueous solution, vortexed for a certain time, nanoparticles were obtained by sonication. During the operation of the two-phase mixing method, the formation and stabilization of the nanoparticles were constrained by five factors: A. volume ratio VsiRNA/VCLD; B. concentration ratio of materials (N/P) (N denotes the protonatable number of N, P the amount of phosphorate residue contained in the siRNA); C ultra sonicating temperature; D ultra sonicating time; E. vortex time. Therefore, to explore the optimal combination of conditions for a two-phase mixing process, four parameters were to be set for each factor:
A. A₁: 5/1, A₂: 10/1, A₃: 15/1, A₄: 20/1
B. B₁: 3/1, B₂: 5/1, B₃: 7/1, B₄: 10/1
C. C₁: 40° C., C₂: 50° C., C₃: 60° C., C₄: 70° C.
D. D₁: 10 min, D₂: 20 min, D₃: 30 min, D₄: 40 min
E. E₁: 20 s, E₂: 40 s, E₃: 1 min, E₄: 2 min

Design for the L₁₆ (4⁵) orthogonal experiments. A total of 16 parallel experiments were conducted. Effects of each factor on the Poly Dispersity Index (PDI) were investigated to determine the optimal preparation formulation.

Experimental operations: A certain amount of CLD solid powder was accurately weighed out, dissolved in absolute ethanol to a final concentration of 1.142 mg/mL (100 μM), stored at 4° C. siRNA was hydrated using DEPC water to a final concentration of 10 μM (spared). The CLD ethanol solution was instilled into the aqueous solution of siRNA according to different volume ratio (A) and different N/P ratio (B), vortexed and mixed the solutions for a certain time (E). After that, the products were prepared at different ultra sonicating temperature (C) and different ultra sonicating time (D), after the completion of the operations particle sizes and potentials were determined.

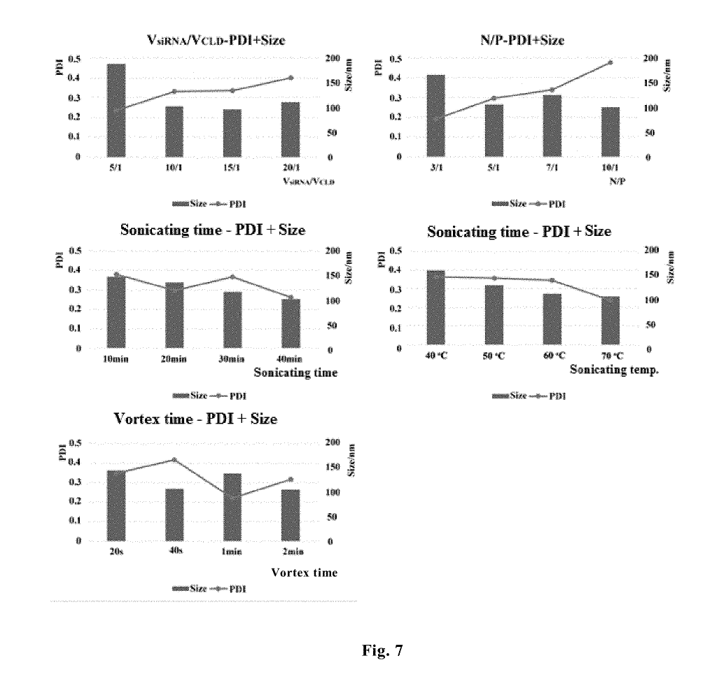

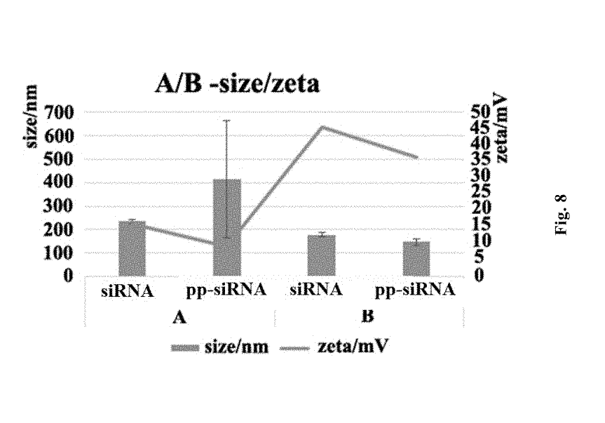

Results and analysis (FIG. 7): PDI was used as the benchmark, by analysis of five factors, two optimal combination of conditions A and B could be obtained, namely: volume ratio of V_{SiRNA}/V_{CLD}=5/1, N/P=3/1 (A) and 5/1 (B), the ultra sonicating temperature was 70° C., the ultra sonicating time was 40 min and the vortex time was 1 min. Under such formulation process conditions, 3′, 3″-bispeptide-siRNA conjugate and CLD could form stable nanoparticles (FIG. 8), and optimal nanocomposite particles could be obtained.

### Embodiment 6. Evaluation of Properties of Four Kinds of Formulations of Natural- and 3′, 3″Bipiypeptide-siRNA Conjugates Bound with Cationic Liposome Vector CLD (Serum Stability Test, Erythrocyte Hemolysis Test and Dilution Stability Test) In Vitro

Procedures: Four formulations of native- and 3′,3″-bispeptide-siRNA conjugates bound with the cationic liposome vector CLD were prepared according to Embodiment 4 and Embodiment 5.

Serum stability test: 100 μL of different preparations and 5% glucose solutions were added into 96-well plate, mixed with 100 μL fetal bovine serum, incubated at 37° C. for 0 min, 5 min, 10 min, 30 min, 1 h, 3 h, 5 h, 10 h, 24 h, 33 h, and 48 h, respectively. The absorbance (OD value) at 630 nm was determined using Bio-Rad microplate reader. Three replicates were carried out for each sample.

Erythrocyte hemolysis test: Blood samples were collected from venous plexus of SD rats' eyes, centrifuged at 1500 g at 4° C. for 10 min, the serum was discarded. The isolated red blood cells obtained were washed with 0.9% physiological saline, suspended in pH 7.38 phosphate buffer solution to prepare 2% (v/v) red blood cell suspension 100 μL of the blood cell suspension was instilled into 96-well plate, 100 μL PBS (negative control), 1% Triton X-100 (positive control) or sample solutions with series of concentrations were added to each well, and incubated for 1 h at 37° C. Intact red blood cells were removed by centrifugation, and absorbance at 540 nm was measured with a Bio-Rad plate reader. The relative hemolytic rate was calculated using the following formula:

([Abs]ₛₐₘₚₗₑ−[Abs]_{buffer})/([Abs]_{Triton X-100}−[Abs]_{buffer})×100%.

Experiments on dilution stability: Four preparations of formulations prepared with siRNA (200 μL, 2 μM)/CLD (40 μL, 50 μM) were diluted 5, 10, 20, 40 times, respectively, to observe their characteristics changes (for the measurement of potentials, diluted 5 more times).

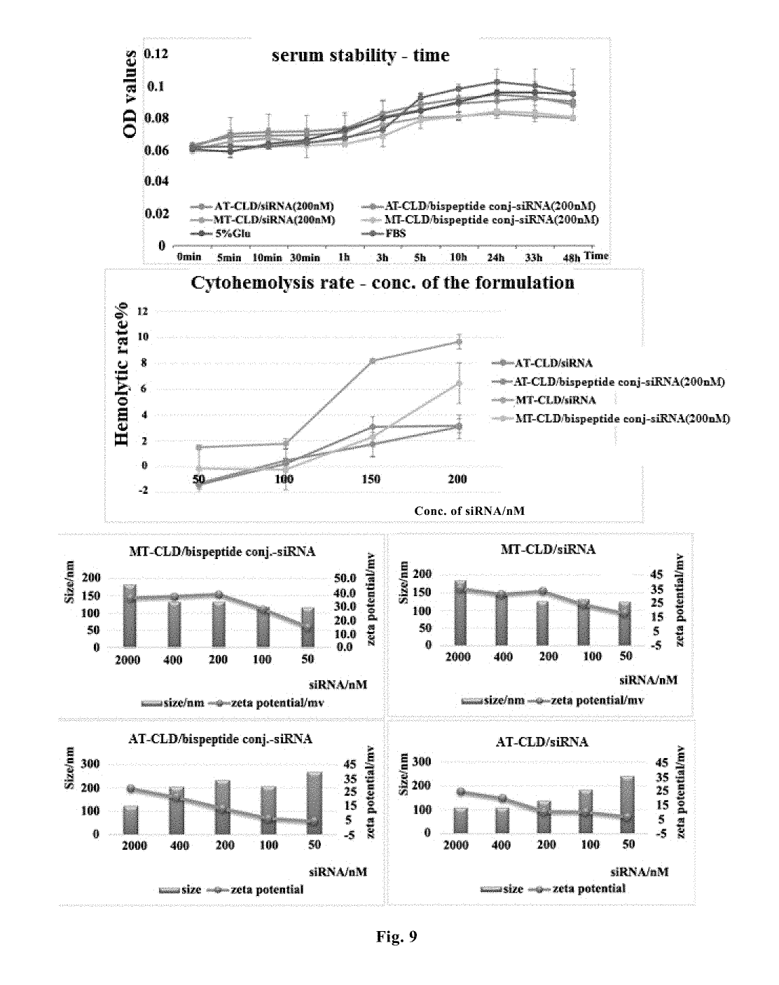

Results: From FIG. 9, it can be seen that there was no significant difference between the absorbance values of the four preparation groups and the FBS group. This showed that the four preparations did not agglutinate with albumin in the serum and were very safe; the hemolysis rate of the natural siRNA/CLD complex prepared by the MT method was the highest, which was related to the highest surface electric potential. In addition, the hemolysis rate of MT preparations was relatively higher than those of AT method preparations, which was related to the assembly manner, namely: the siRNA was mainly in the inner layer in the MT method preparations, whereas in the AT method preparations the siRNA was more in the outer layer. Therefore, hemolysis rates would be different. As a whole, the hemolysis rate of four formulations were all relatively low, thus they were relatively safe; a comparison of the products by the two preparation methods showed that, in the dilution process, those from AT method changed more in potentials and particle sizes, indicating that those from MT method had obviously higher diluted stability than those from AT method, which also showed that in MT method the interaction between CLD and whether natural siRNA or 3′,3″-bispeptide-siRNA conjugates were all stronger than in AT method. Between all the preparations produced from MT method, formulations prepared from the 3′,3″-bispeptide-siRNA conjugates had a lower diluted surface electric potential than those from native siRNAs.

### Embodiment 7. Dynamic Light Scanning (DLS) Diagrams of Formulations Formed from Natural-siRNA as Well as 3′, 3″-Bispeptide-siRNA Conjugates Bound with Cationic Liposome Vector CLD

Experimental Procedures: Four formulations formed by incorporation of native-siRNA or 3′,3″-bispeptide-siRNA conjugates bound with cationic liposome vector CLD were prepared as in Embodiment 4 and Embodiment 5. A certain amount of aqueous solutions of the products were added into EP tubes and the respective parameters such as hydrated size, surface electric potential and polydispersity were measured using a dynamic light scattering instrument (Zetasizer Nano ZSP).

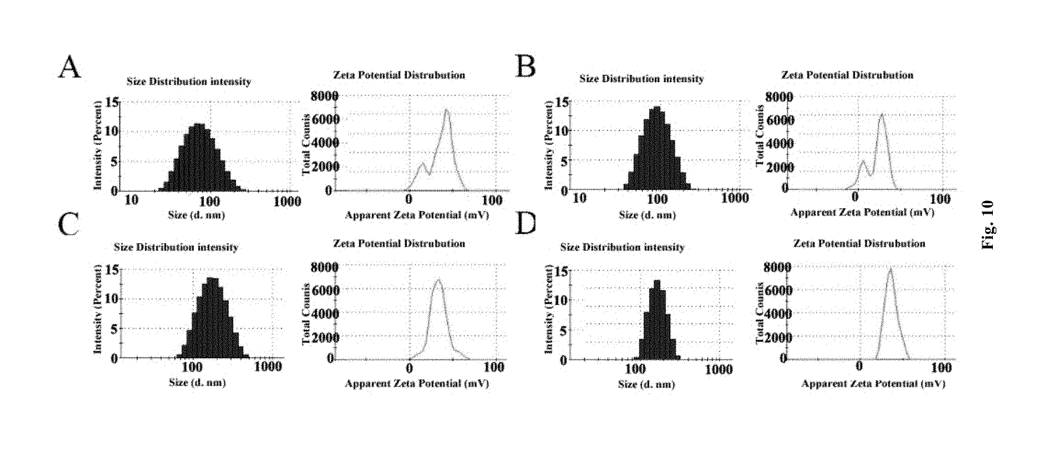

The experimental results (shown in FIG. 10 and Table 2) showed that after exploration and investigation of the preparation process conditions, at present two preparation processes all could produce relatively uniform, stable spherical-like composite nanoparticles. The polydispersity indices (PDI) of all four formulations were less than 0.3, indicating that the products formed had good dynamic profiles. The surface electric potentials of the four preparations were all controlled within a range of +20-30 mV, and the surface electric potentials in this range favored the efficient uptake of the formulation by the cells while minimizing the cytotoxicity posed by the positive charge. Among them, the surface electric potentials of the preparation formed from 3′, 3″-bispeptide-siRNA conjugate/CLD was lower than that of natural siRNA/CLD produced in the same preparation process, this, on top of the effective uptake of cells, 3′, 3″-bispeptide-siRNA conjugates had lower cytotoxicity and being able to exert their silencing activity more safely. As far as hydrated particle size was concerned, the particle size could be controlled to about 100 nm to 150 nm by the described process, and particles of this size had a good passive targeting effect in vivo, that is, enhanced permeability and retention effect (EPR). In the spin-membrane hydration method (AT method), nanocomposites with larger particle sizes and lower surface electric potentials could be formed from 3′,3″-bispeptide-siRNA conjugates and the cationic liposome vector CLD. Above-mentioned features were significant differences from features of the assembled systems obtained with natural siRNA, therefore would lead to the specificity of the biological behaviors in the later stage.

TABLE 2

| | Mean particle size | Polydispersity | Zeta potential |
|---|---|---|---|
| Preparations | (d, nm) | Index(PDI) | (mV) |
| AT-pp-siRNA | 100.06 ± 2.96 | 0.200 ± 0.046 | 22.40 ± 3.50 |
| AT--siRNA | 85.83 ± 7.90 | 0.212 ± 0.012 | 35.53 ± 1.97 |
| MT-pp-siRNA | 150.20 ± 7.60 | 0.140 ± 0.063 | 22.97 ± 3.14 |
| MT-siRNA | 151.37 ± 12.50 | 0.166 ± 0.016 | 28.00 ± 2.46 |

### Embodiment 8. Supramolecular Structure Characterization of Cationic Liposome Vector CLD Combined with Native siRNAs and 3′, 3″Bipiypeptide-siRNA Conjugates (AT Method)

Experimental procedures: The internal structure and apparent morphology diagram of composite particles formed with CLD liposomes and natural siRNA (siMek1) as well as 3′,3″-bispeptide-siRNA conjugates (PA/PS-siMek1, prepared using the method in Embodiment 4) were collectively investigated with transmission electron microscopy (TEM) and atomic force microscope (AFM). The operation was as follows: first, the preparation according to the procedure of Embodiment 4 of the nanostructures formed with liposomes only, natural siRNA and CLD as well as 3′,3″-bispeptide-siRNA conjugate, respectively. 4 μL of three kinds of composite aqueous solution, respectively, were instilled onto the square hole copper nets (model GilderGrids 400 mesh) and evaporated naturally at room temperature to dryness. The internal structure of the samples was observed with TEM (model: Philips Tacnai G2 20 S-TWIN Microscope operating at 200 kV) to obtain the TEM images of the three samples. Instil 1 mL of three kinds of composite aqueous solutions onto 1 cm² quartz coverplate, the solvent was dried at room temperature, and then an AFM (Model: Multimode IIIa AFM Veeco Metrology, USA) equipment was used for imaging and analysis of their apparent morphology diagrams.

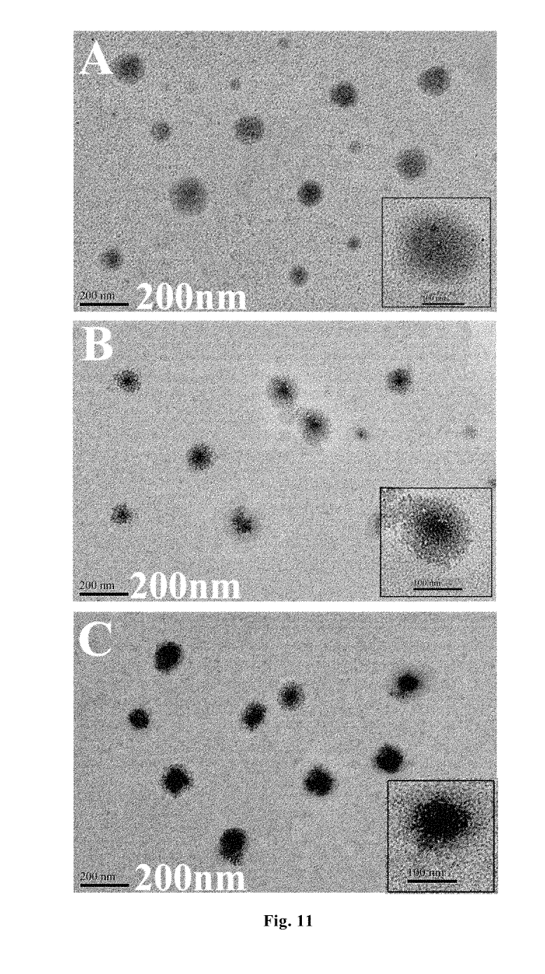

Results: FIG. 11 shows the TEM results of three configurations of sample particles of non-conjugated cationic liposome vector, composite formed by cationic liposome vector CLD and natural siRNA, as well as composite formed by cationic liposome vector CLD and 3′,3″-bispeptide-siRNA conjugate. It can be seen that non-conjugated cationic liposome vector particles showed a spherical shape, they were typical spherical lipid vesicles. When the cationic liposome vector CLD bound to natural siRNA and 3′,3″-bispeptide-siRNA conjugates, respectively, the structural morphologies of the lipid particles showed obvious changes: in FIG. 11B, in the center portion, the color is darker, whereas in the edge portion the color was relatively shallower. This shows that after complex formation, the native siRNA could be effectively encapsulated by the cationic liposome vector CLD, leaving the siRNA in the inner space of the complex and thereby being protected by the cationic liposome vector CLD. It can be seen from FIG. 11C that the apparent morphology diagram of the composite formed by cationic liposome vector CLD entrapping the 3′,3″-bispeptide-siRNA conjugate was significantly different from the above two: the last composite showed formation of darker, spherical structure-like particles, and no obvious marginal sharrow space (FIG. 11B). When a positively charged cationic liposome vector particle interacts with a negatively charged siRNA, the siRNA will be compressively bound to the lipid particle surface due to the interaction between the charges. This compression will inevitably form a compressive layer of siRNA on the surface of the lipid particles, forming a darker color rim on the surface of the lipid particles. According to the working principle of TEM imaging it can be deducted that these particles would be compressed more closely. Therefore, it can be seen from the TEM results that the composites formed by the 3′, 3″-bispeptide-siRNA conjugate with the cationic liposome vector CLD is more tightly compressed in comparison with the native siRNA formed composites, and this phenomenon is due to the 3′-terminally covalently conjugated peptide sequence.

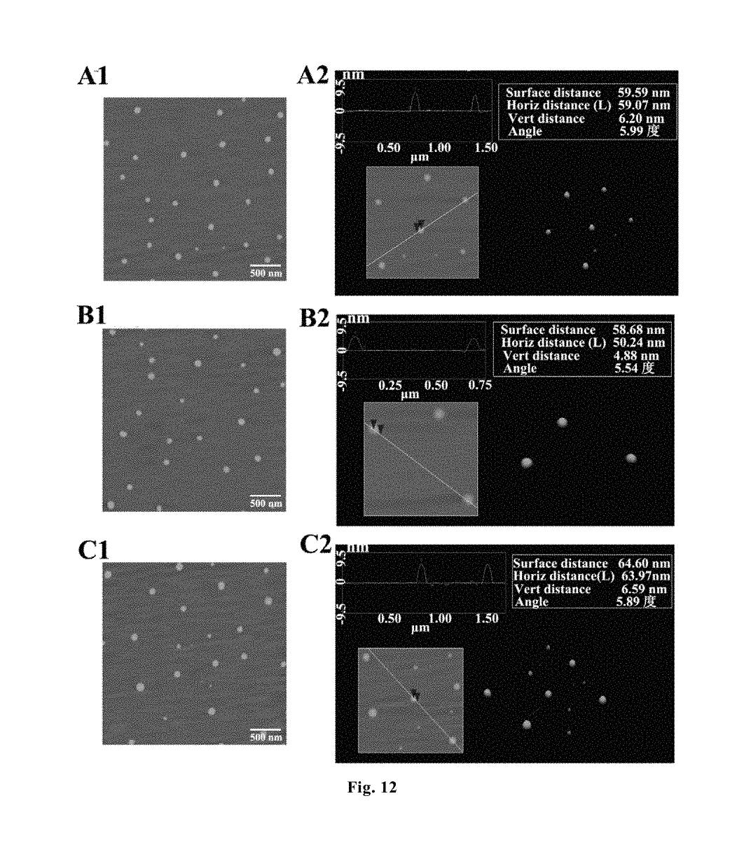

The AFM results from the composite formed by the interaction of cationic lipid particles with native siRNA (siMek1) as well as 3′,3″-bispeptide-siRNA conjugate (siMek1) showed that the two demonstrated round shapes and uniform distributions (FIG. 12). This result was consistent with the TEM results. When the experimental conditions were the same (N/P=20, msiRNA=200 ng), the binding morphology diagram of the cationic liposome vector binding to the 3′,3″-bispeptide-siRNA conjugates was different from that of the cationic liposome vector with natural siRNA. As can be seen from FIG. 12A, the cationic liposome vector CLD had a horizontal distance (ie, diameter) of about 120 nm and a vertical distance of about 6 nm. The difference between the vertical distance and the horizontal one is because that the sample preparation method used the solvent-evaporation method, by dropping a certain concentration of the material solution onto the mica sheet, the solvent was volatilized before detection. During the process the particles collapsed down under the influence of gravity, so that the vertical distance was lowered. FIGS. 12B1 and B2 demonstrated that the composites formed by cationic liposome vector CLD with native siRNA were approximately 100 nm in diameter and the vertical distances were approximately 5 nm. Due to electrostatic adsorption between the cationic liposome vector CLD and the native siRNA, the cationic liposome vector CLD was further compressed, so that the size of the composites formed was slightly smaller than that of the cationic liposome vector CLD. FIG. 12C shows that the particle size of the composite formed by the cationic liposome vector CLD and the 3′,3″-bispeptide-siRNA conjugate was about 130 nm with a vertical distance of about 6.6 nm. The particle size and vertical distance were larger than that of the composite formed by natural siRNA and cationic liposome vector CLD. According to this phenomenon and the TEM data, the following conclusions could be drawn: the interaction force between 3′,3″-bispeptide-siRNA conjugates and the cationic liposome vector CLD was larger than the pure electrostatic force between the natural siRNA and the cationic liposome vector CLD, so that the composite formed by the cationic liposome vector compresses more tightly and simultaneously forms more intercalated layers, increasing the particle size of the single composites. There was a difference between the composite formed by native siMB3 and the cationic liposome vector CLD, and that by the 3′,3″-bispeptide-siRNA conjugates and the cationic liposome vector CLD. This difference reflected the fact that the bispeptide-bound siRNAs can be more closely linked to the cation, a phenomenon that resulted from the peptide sequence covalently conjugated at the 3′-end. In summary, due to the introduction of the terminal peptide, the action mode of the 3′,3″-bispeptide-siRNA conjugate with the cationic liposome vector CLD to form a supramolecular composite was significantly different from that of native siRNA with the cationic liposome vector CLD. The chemical and biological implications of this difference will be combined with the results of subsequent biological activity evaluation on cationic liposome vector/native siRNA and cationic liposome vector/3′,3″-bispeptide-siRNA conjugates for further analysis.

### Embodiment 9. Cellular Uptake of Four Formulations Formed by Cationic Liposome Vector CLD with Native siRNA as Well as 3′,3″-Bispeptide-siRNA Conjugate

Experimental Procedures: Four formulations of native-as well as 3′,3″-bispeptide-siRNA-conjugates incorporated with the cationic liposome vector CLD were prepared according to Embodiment 4 and Embodiment 5. Melanoma A375 cells was inoculated into 6-well plates at 300,000 cells/well and cultured for 24 hours for cell adherence to the wall, incubate the cells at 37° C. for 15 minutes. Dilute ten-fold with OPTI-MEM, resulting in a final concentration of 100 nM of the bispeptide siRNA and native siRNA. The medium was then discarded and washed twice with 1 mL PBS. 2 mL of the aforementioned preparation of 3′,3″-bispeptide-siRNA and natural siRNA labeled with Cy3 was added to each well and cultured for 4 h. Afterwards, the uptake of the preparations was detected using flow cytometry.

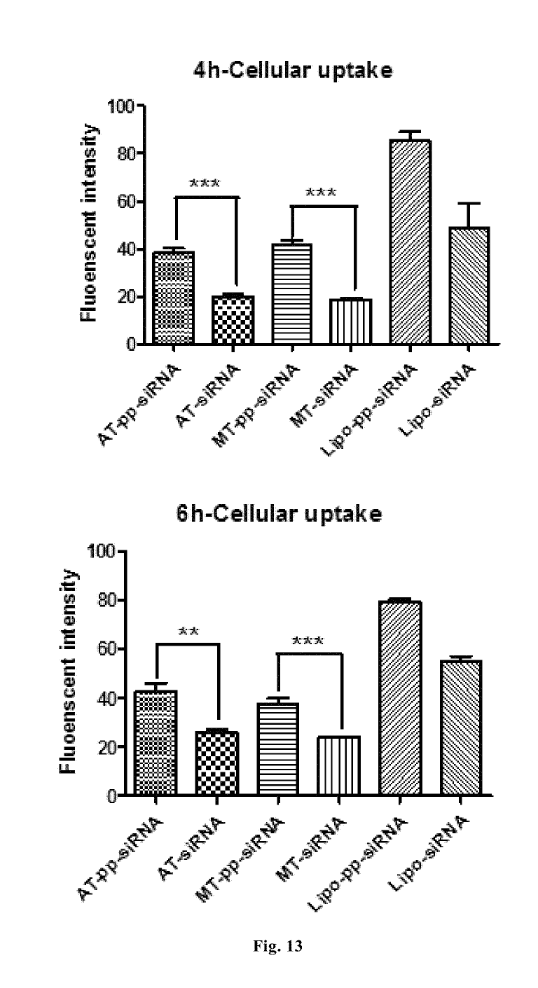

Results (FIG. 13): It can be seen that there was a significant difference in the uptake of 3′,3″-bispeptide-siRNA conjugates/CLD composites and native siRNA/CLD composites by melanoma A375 cells. Whether it was at 4 h or 6 h after dosing the cellular uptake of 3′,3″-bispeptide-siRNA conjugate/CLD composites was significantly higher than that of native siRNA/CLD composites prepared under the same conditions of described process. However, under the same siRNA conditions, there was no significant difference in cellular uptake between the two preparation methods. These results suggested to some extent that the cell transmembrane pattern of 3′,3″-bispeptide-siRNA conjugates was different from that of native siRNAs.

### Embodiment 10. Cellular Uptake of Four Formulations Formed by the Binding of Cationic Liposome Vector CLD to Native siRNA as Well as 3′,3″-Bispeptide-siRNA Conjugate after Treatment with Inhibitor

1. Cell Pathways and the Choice of Appropriate Inhibitors

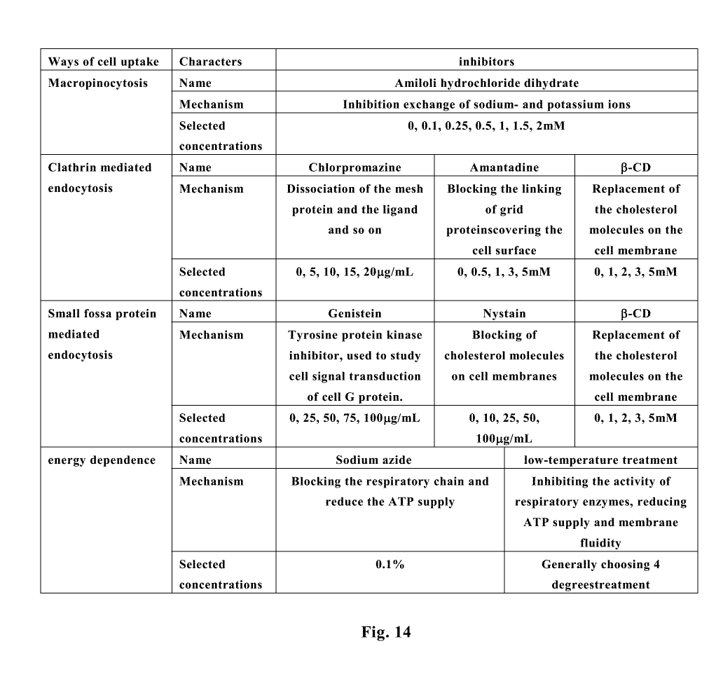

Cellular uptake of exogenous substances is mainly divided into the following ways: phagocytosis, specific receptor-mediated cell, giant drink, clathrin-mediated endocytosis and caveolin-mediated endocytosis. Most of the exogenous substances that enter the cells via these pathways undergo the lysosomal acidification process and are eventually degraded or excreted. Recent studies have shown that in these cell pathways, exogenous substances can be directly released into the cytoplasm when they enter the cell by means of caveolin-mediated endocytosis and to a certain extent can avoid lysosomal degradation. In addition, although the macrophage pathway does go through the lysosome process, but its escape rate is relatively fast. Therefore, when a drug enters the cell by means of the caveolin-mediated endocytosis or macropinocytosis, it is favorable for it to realize its biological activity efficiently. The authors of the present invention took endocytosis as the focus of study, design and study four ways into the cell, namely: macropinocytosis, clathrin-mediated endocytosis, caveolin-mediated endocytosis and energy-dependent endocytosis; and organize the relevant research data to sum up the small molecule inhibitors corresponding to each entry pathway (FIG. 14).

2. Investigation on Small Molecule Cell Pathway Inhibitors on the Cellular Uptake of Bispeptide siRNA and Natural siRNA

Experimental operation: 300,000 per well Human melanoma A375 cells were inoculated into 12-well plate. After culturing for 24 h, the original medium was washed and washed twice with 1 mL pre-cooling PBS and added with different concentrations of inhibitors at 2 mL/well (FIG. 14), incubated under 5% CO₂ for 30 min at 37° C. Afterwards, different CLD/Cy3-siRNA composites (Formulation operations as in Embodiment 4, 5) were added 200 μL/well, and the final Cy3-siRNA concentration of 100 nM. After incubating at 37° C. and 5% CO₂ for 6 h, thecontents were digested with 300 □L trypsin at room temperature for 3 min, added with 1 mL medium, stopped digestion, thecontents were aspirated into 2 mL centrifuge tubes, discarded the medium. 1 mL of pre-cooled PBS was added to wash the cells twice, resuspended the cells into 400 μL PBS, the fluorescence intensities of Cy3 in the cells were measured using a flow cytometer, 10,000 cells were collected, the excitation wavelength was 550 nm and emission wavelength 590 nm. The collected data were analyzed using FCS Express V3 software.

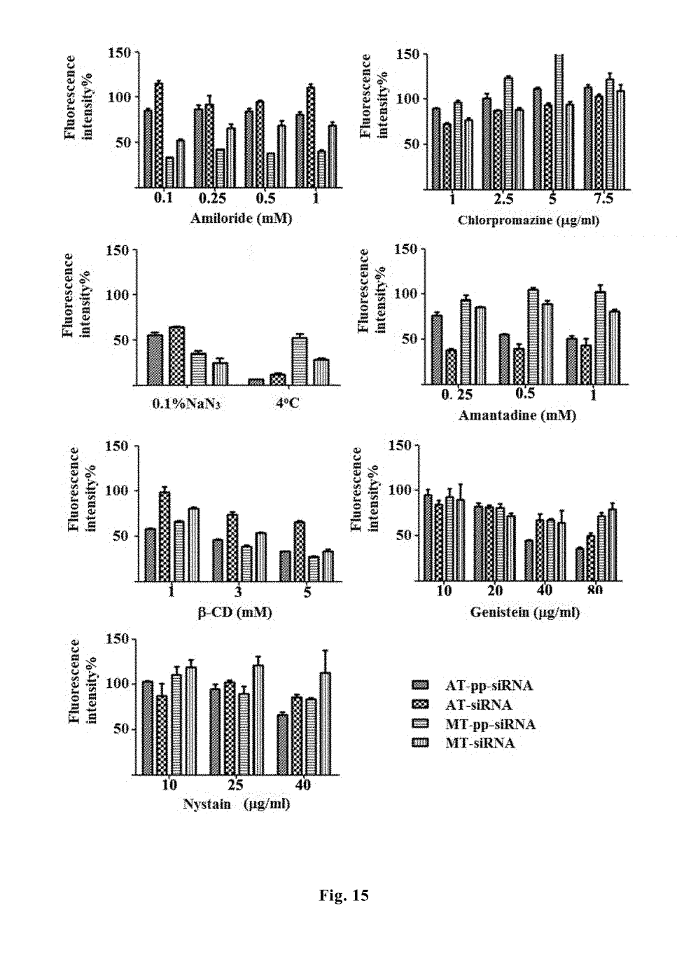

Results (shown in FIG. 15): As for the macropinocytosis pathway, the two prepared by the two-phase mixing method (MT method) showed higher endocytosis proportions relative to the two formulations prepared by the spin-membrane hydration method (AT method), of which the 3′,3″-bispeptide-siRNA conjugates obtained by MT method showed the largest proportion. As for the clathrin-mediated endocytosis, natural siRNA prepared by the spin-membrane hydration method (AT method) showed the largest proportion in the amantadine channel. In addition, after inhibition the cell channel using chlorpromazine, cell uptake increased instead, indicating that inhibition of this pathway alone would activate other pathways to increase cellular uptake of siRNA. With respect to the caveolin-mediated endocytosis pathway, the proportion of 3′,3″-bispeptide-siRNA conjugates formed by the same formulation process conditions (whether it was AT or MT method) was greater than that of natural siRNA conjugates. Among the ATP-dependent pathways, all four preparations had greater dependence on that pathway, with no significant difference between them.

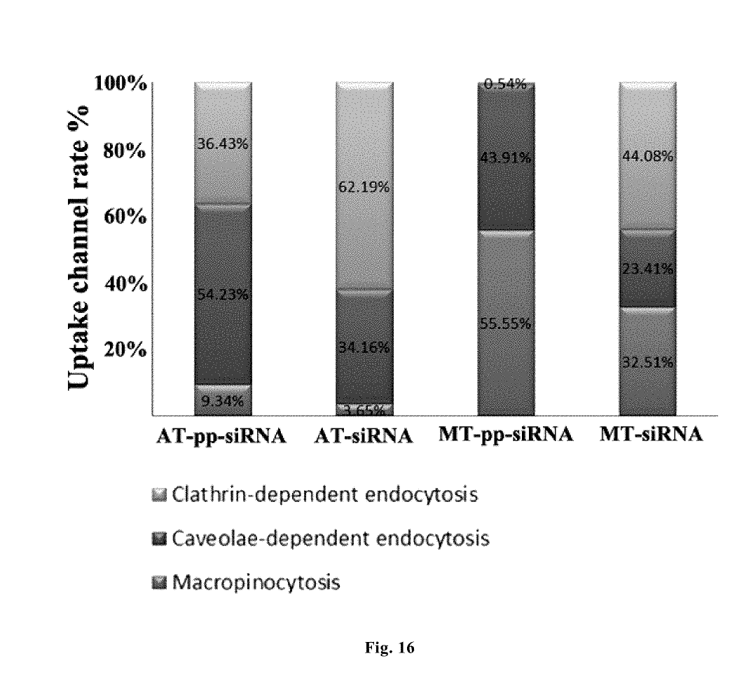

After the study on endocytosis mechanism of the four preparations it can be summarized as follows (FIG. 16): 1) cellular uptake of four kinds of preparations was not through a single pathway; 2) under the same formulation conditions (whether it was AT or MT method), the ratio of cellular uptake of 3′,3″-bispeptide-siRNA conjugates relying on caveolin-mediated endocytosis was significantly higher than that of native siRNA conjugates, whereas the reticulin-mediated endocytosis ratio was significantly lower than Natural siRNA; 3) the cellular uptake proportions of 3′,3″-bispeptide-siRNA conjugate and native siRNA conjugate prepared by the spin-membrane hydration method (AT method) through macropinocytosis-dependent pathway was significantly lower than formulations prepared by the two-phase mixing method (MT method); 4) From the conclusions 2) and 3), it can be inferred that the formulations prepared by the two-phase mixing method (MT method) would have more chance to bypass the lysosomes (ie, caveolin-mediation and macropinocytosis would cause the siRNA to bypass lysosomes or realize rapid lysosomal escape), and the difference between 3′,3″-bispeptide-siRNA conjugate and native siRNA produced composites produced with the two-phase mixing method (MT method) was significantly higher than that produced with spin-membrane hydration method (AT method), which would result in larger differences in gene silencing effects.

Therefore, through the exploration and optimization of formulation process conditions, a uniform and stable supramolecular assembly formed by natural siRNA as well as 3′, 3″-bispeptide-siRNA conjugate and cationic liposome vector CLD can be obtained, they would further be able to regulate the endocytosis pathways of 3′,3″-bispeptide-siRNA conjugates, allowing them to exert the RNAi effect more efficiently. At the same time, the silencing activities of 3′,3″-bispeptide-siRNA conjugates as well as natural siRNAs can also be speculated based on the analysis of the transmembrane mechanism: 1) both with MT- and AT method, 3′, 3″-bispeptide-siRNA conjugates had higher silencing activity than native siRNAs (since the ratio of 3′, 3″-bispeptide-siRNA conjugates through the caveolin-mediation and macropinocytosis mediated endocytosis was relatively higher than that of native siRNA); 2) the silencing activity of 3′,3″-bispeptide-siRNA conjugate/CLD complex prepared by MT method was the best among the four preparations (because the ratio of caveolin and macrophage endocytosis pathways were 99%). 3) The silencing activity of natural siRNA/CLD composite prepared by AT method was the worst (because of the highest endocytosis proportion was through clathrin mediated pathway, which meant that more siRNA could be degraded by lysosomes).

### Embodiment 11. RT-PCR Results of Silencing Activity of Four Preparations Formed by Cationic Liposome Vector CLD Bound to Native siRNA as Well as 3′,3″-Bispeptide-siRNA Conjugate at Different Times and at Different Concentrations

Experimental Procedures: Four formulations of 3′,3″-bispeptide-siRNA conjugates and native siRNA with the cationic liposome vector CLD were prepared as in Embodiment 4 and Embodiment 5. 100,000 per well Human melanoma A375 cells were added into 6-well plate, cultured overnight. 5 μL CLD were mixed with modified siRNA at a final concentration of 30 nM, 60 nM, and 100 nM, respectively. The mixtures were incubated at room temperature for 15 min, the cells were add into the culture plate and incubated for 24 h. Total RNA were extracted at different time points (24 h, 48 h, 72 h) with TRizol, reverse transcribed into cDNA. Real-time experiments were conducted with Gotaq Green Mix to investigate the siRNA gene silencing effect on mRNA level. The total protein was extracted and quantified using BSA as benchmark. The proteins were separate by means of polyacrylamide gel (separation gel 10%, concentration gel 5%), subsequently, after transferring the proteins from the gel, carry through primary and secondary antibodyimmunal reaction. Detect protein knockout effect by using Biorad chemiluminescence gel detection system.

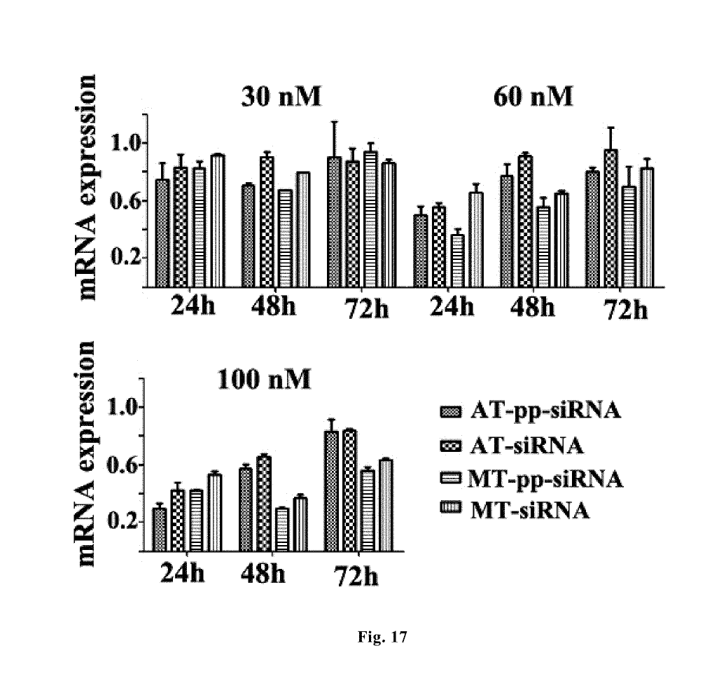

Results (FIG. 17): Four formulations showed better silencing effect at 60 nM and 100 nM dosing with significant differences. Consistent with cellular uptake, at these two concentrations, the 3′,3″-bispeptide-siRNA conjugate preparations produced by the MT method showed better silencing efficacy than the other three formulations with significant differences. With respect to the difference between two preparative methods, the silencing activities of the two preparations produced with the MT method on the target mRNA was better than those of the corresponding two preparations with the AT method. In addition, the silencing effects of the four formulations increased in a concentration-dependent manner.

The experimental results of the silencing effect were consistent with our predictions based on the transmembrane pathway. This shows that we can control the transmembrane pathway of the nanocomposite formed by the 3′,3″-bispeptide-siRNA conjugates and the cationic liposome vector CLD to predict and judge the later stage silencing activity, change the intracellular metabolic behavior of siRNA to some extent, to achieve high efficiency of gene silencing.

### Embodiment 12. The Investigation on Biological Activity of Isonucleoside Co-Modified 3′,3″-Bispeptide-siRNA Conjugates/Cationic Liposome Vector CLD Using Real-Time Experiment

Preparation of isonucleoside co-modified 3′,3″-bispeptide-siRNA conjugates:

The pre-modification siMek1 sequence was as follows:

siMek1:

| | |
|---|---|
| sense strand: | 5′-GCAACUCAUGGUUCAUGCUdtdt-3′; |
| antisense strand: | 5′-AGCAUGAACCAUGAGUUGCdtdt-3′ |

Peptide fragment of Formula III (PA/PS-siMek1) was conjugated to the 3′ terminus of the sense and antisense strands of the siMek1 sequence described above;

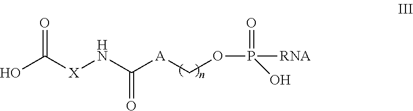

Wherein X is a 6-peptide sequence H-Leu-Ala-Leu-Leu-Ala-Lys-OH (KALLAL), A is a carbon atom and n is 1.

Meanwhile, the first nucleotide of the 5′ end of sense strand of the siMek1 sequence was conjugated with an isonucleotide (D1PP) of Formula I, or the first nucleotide of the 5′ end of the sense strand of the siMek1 sequence was conjugated with the isonucleoside of Formula II (L1PP).

Experimental procedures: 200,000/well Hela cells were added into 6-well plates and transfected after incubated for 12-14 h. Under the condition of the same siRNA concentration (30 nM), 5 μL commercial transfection reagent RNAiMAX, and 2.6 μL cationic liposome vector (at a concentration of 6.3 mg/mL) were added. The siRNA was mixed with transfection reagent and incubated at room temperature for 25 min, the mixture was added to the culture plate and cultured for 24 h. The total RNA was extracted using TRizol, and reverse transcribed into cDNA. Real-time experiments were performed with Gotaq Green Mix to investigate the gene silencing effect of siRNA at the mRNA level.

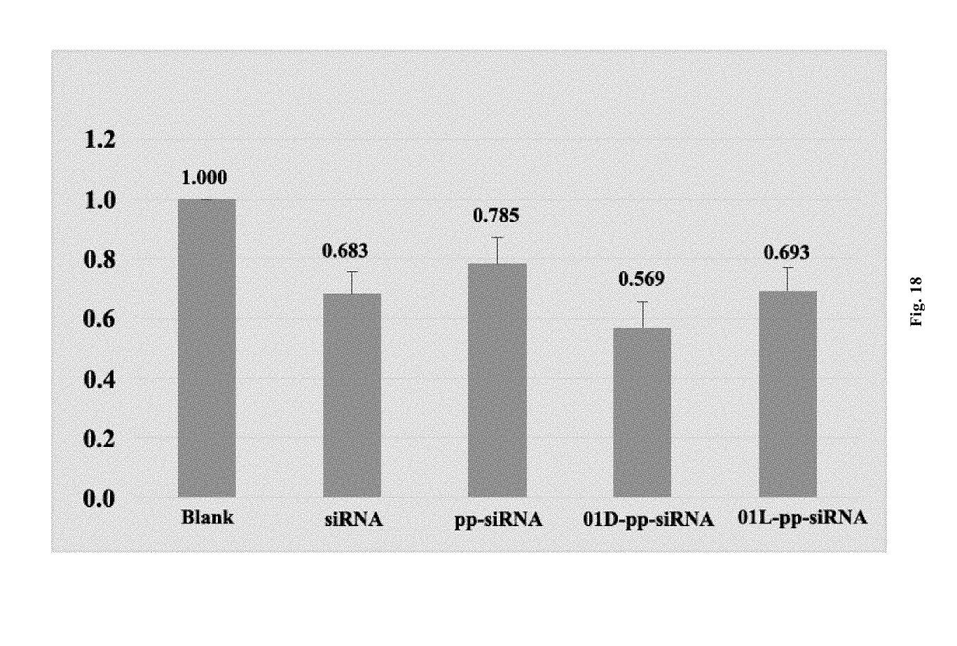

The results of real-time PCR showed that silencing of target mRNA by 3′3′-bispeptide-siRNA conjugate co-modified with cationic DNA vector CLD and isonucleoside (FIG. 18) showed that, compared with the silencing effect of the control group, the cationic liposome vector CLD conjugated with natural siRNA, as well as with 3′,3″-bispeptide-siRNA, and 3′, 3″-bispeptide-siRNA co-modified by isonucleoside, the compounds could all effectively inhibit the corresponding target mRNA. Wherein the 3′,3″-bispeptide-siRNA conjugates had a comparable silencing effect of natural siRNA under the condition that the cationic liposome vector CLD acts as a conjugating reagent, indicating that under the conditions that the silencing activity is not affected, the bispeptide can be more effective in improving serum stability and reduce the degradation of siRNA by ribozyme. In addition, the 3′,3″-bispeptide-siRNA conjugates modified with isonucleosides had a better silencing activity than the 3′, 3″-bispeptide-siRNA conjugates, indicating that on the basis of improving stability, the strategy of co-modifying with isonucleoside and with bispeptide could significantly improve its silencing ability to the target. It can be seen in the figure that the 3′,3″-bispeptide-siRNAco-modified with d-isonucleoside and with the cationic liposome vector CLD had the best silencing activity. This showed that the joint modification of isonucleoside with peptide conjugation, as well as cationic liposome vector-incorporated siRNA had a good application prospect and a value to be continuously development.

### Embodiment 13. Investigation on the Stability of Formulations of Isonucleoside Co-Modified 3′,3″-Bispeptide-siRNA Conjugate/Cationic Liposome CLDs as Well as Natural siRNA/Cationic CLD Using Gel Electrophoresis

Experimental Procedures: The preparation of 3′,3″-bispeptide-siRNA conjugates/CLD formulations co-modified with isonucleosides is described in Embodiment 4. Polyanion exchange experiment were performed to investigate the stability of 3′,3″-bispeptide-siRNA conjugate/cationic liposome CLD co-modified with isonucleoside. The polyanion selected for this experiment was heparin. Heparin is a sulfonated mucopolysaccharide. A variety of acidic proteins exist in the human body both in the intracellular- and in the extracellular environment. These proteins are negatively charged and can compete with siRNAs to disrupt the stability of the siRNA/vector system. If siRNA in the vector composite is replaced by heparin at a slower rate and to a lesser extent, this indicates that the siRNA/vector composite is stable, which increases the in vivo stability of the composite and facilitates better silencing activity.

CLD liposomes were prepared following the procedures of Embodiment 4. The isonucleoside co-modified 3′,3″bispeptide-siRNA conjugate/CLD composite and native siRNA/CLD nanocomposite were prepared in a ratio to form a stable formulation. Different preparations were mixed with 0.1, 0.2, 0.3, 0.4, 0.5, 0.75, 1.0, 1.5, 3.0 IU heparin/μgsiRNA, respectively. After incubating at 37° C. for 30 min, 5× loading buffer was added, The EB/siRNA fluorescence was observed by conducting electrophoresisat 80 V for 3 min on 1% agarose gel containing 0.5 μg/mL EB, and then at 100 V for 15 min. EB/siRNA fluorescence was observed by gel imaging system. Free siRNA was used as control.

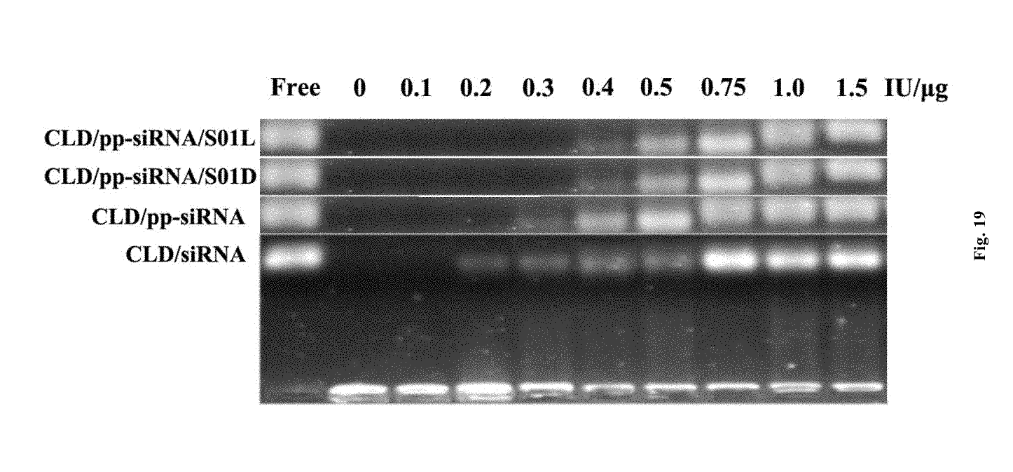

Results of the study showed that the stability of the composite formed by natural siRNA and cationic liposome CLD was the worst, (FIG. 19) since the natural siRNA started to show dissociation under the condition of 0.2 IU/g heparin, indicating that natural siRNA was the first to be replaced with polyanionic heparin; in the case of the 3′,3″-bispeptide-siRNA conjugate, a slight dissociation started to occur at a heparin concentration of 0.3 IU/μg, indicating that the introduction of a bispeptide at the end of the siRNA enhanced its interaction with the cationic liposome CLD, so that the stability of the entire system compared with that produced by natural siRNA was obviously improved; whereas in the case of isonucleoside co-modified 3′, 3″-bispeptide-siRNA, the incorporation of d-isonucleoside and 1-isonucleoside alike would further enhanced their interaction with CLD, but the enhancement was not significant (the stabilities were similar to 3′, 3″dippeptide-siRNA conjugate). This is because the interaction between the nucleic acid drug and the CLD vector relies mainly on electrostatic adsorption and a certain hydrophobic effect, whereas the incorporation of the isonucleoside caused no significant change on the electrical properties, because the main purpose of incorporating the isonucleoside into the siRNA was to embody the advantages of subsequent biology. In conclusion, the 3′,3″-bispeptide-siRNA conjugate/cationic liposome CLD co-modified by isonucleoside has better stability.

The information presented and described in detail herein is sufficient to attain the above objects of the present invention and thus the preferred embodiments of the present invention represent the subject of the present invention, which is broadly covered by the present invention. The scope of the present invention fully encompasses other embodiments apparent to those skilled in the art, and thus the scope of the present invention is not limited by any statement apart from the appended claims, wherein the singular forms of the elements stated does not mean “one and only” but rather “one or more.” For the ordinary technician in the art, all the equivalents of structure, composition, and function of above-described, published preferred embodiments and additional embodiments should hereby incorporate this article for reference and are intended to be covered by the claims of the present invention.

In addition, no certain apparatus or method is required to solve each of the problems that the present invention addresses, as they are all included in the claims of the present invention. In addition, no matter whether all the parts, components, or procedures in the disclosed matter of the present invention are explicitly recited in the claims, none of them contributes to the public. However, it is apparent to those skilled in the art that, without departing from the spirit and scope of the invention as set forth in the appended claims, various changes and modifications in the form, reagents and details of synthesis can be made.

1: A method for integrated chemical modification of small interfering RNAs (siRNAs), comprising:
incorporating one or more sites of sense strand or antisense strand of siRNA with D- or L-isonucleosides;
modifying 3′-end of the sense strand and/or the antisense strand of siRNA with peptide-conjugation; and
using cationic liposome vector to realize the transmembrane transport of the modified siRNAs.

2: The integrated chemical modification method according to claim 1, wherein the D- or L-isonucleoside, respectively, has the structure of the following chemical formula: 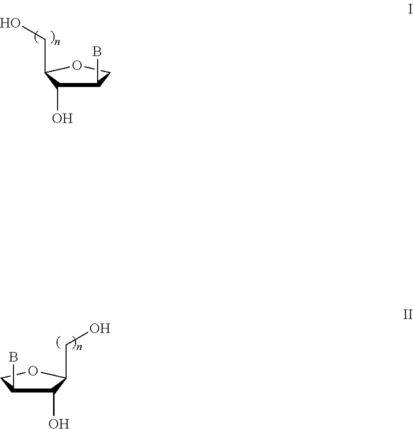
wherein n=1, 2 and 3; B is thymine (T), uracil (U), cytosine (C), guanosyl (G), and adenine (A), the isonucleoside shown in chemical Formula I is a D-isonucleoside, and the isonucleoside shown in chemical Formula II is an L-isonucleoside.

3: The integrated chemical modification method according to claim 1, wherein said peptide conjugation modification is performed on the 3′ end of the sense and antisense strands of siRNA, wherein the general formula of the linkage of the peptide conjugate fragment with the RNA is: 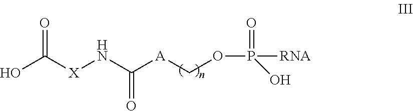
wherein X is a polypeptide sequence, A is a substituted or unsubstituted benzene ring or carbon atom, and n is 0, 1, 2, 3, 4.

4: The synthetic chemical modification method according to claim 3, wherein said X is H-Leu-Ala-Leu-Leu-Ala-Lys-OH.

5: The integrated chemical modification method according to claim 1, wherein the cationic liposome vector is a commercial one, including RNAiMax, Lipofectamine, or a cationic liposome vectors of the Formula IV: 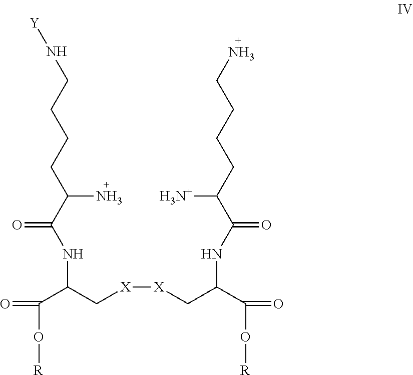
wherein X are sulfur atoms (S) or carbon atoms (C), Y is other amino-containing structure or a targeting group, and R is saturated or unsaturated aliphatic chains or a hydrophobic molecules.

6: The integrated chemical modification method according to claim 5, wherein the unsaturated fatty chain represented by R is oleyl.

7: The integrated chemical modification method according to claim 1, wherein incorporating of the isonucleoside into the siRNA is achieved by solid-phase synthesis, at the corresponding incorporating position, and coupling is carried out with isonucleosidephosphoramidite monomer in place of the natural nucleoside phosphoramidite monomer.

8: The integrated chemical modification method according to claim 7, wherein before performing siRNA modification, the isonucleoside compounds shown in chemical Formula I and/or II are respectively prepared into isonucleosidephosphoramidite monomer shown in chemical formula V and/or VI, incorporation is realized by means of a DNA synthesizer using phosphoramidite method; 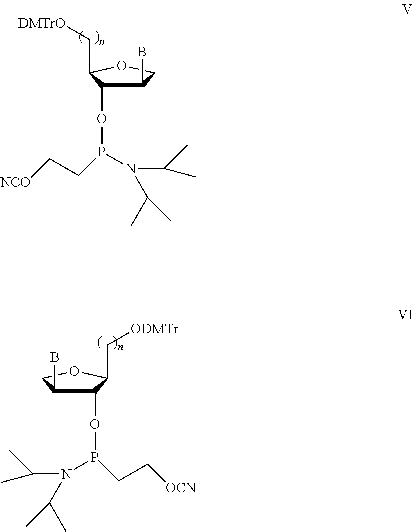
wherein n=1, 2, 3; B is thymine (T), uracil (U) and amino protected guanine (G), adenine (A), cytosine (C).

9: The integrated chemical modification method according to claim 1, further comprising the joint use of two or more chemical modification strategies chosen from a list consisting of: 2′-O-methoxy (2′-OMe), 2′-fluoro (2′-F), locked nucleotides (LNAs), and phosphosulfur skeleton modifications.

10: The integrated chemical modification method as claimed in claim 1, further comprising:
(1) weighting out CLD solid powder and dissolving the CLD solid powder in anhydrous ethanol to obtain an ethanol solution of CLD, preserve at 4° C. until use;
(2) conducting peptide conjugation modification at the 3′-end of the sense strand and antisense strand of siRNA, and using DEPC water to hydrate it;
(3) according to ratios of V_{siRN}A/V_{CLD}=5/1, N/P=3/1 or 5/1, adding the CLD ethanol solution obtained in steps (1) and (2), as well as the siRNA after DEPC water hydration, sonicate for 40 min at 70° C. and vortex for 1 min to obtain nano-composite particles.

11: The integrated chemical modification method as claimed in claim 1, wherein the pathway or proportion of the endocytosis of the nano-composite particles formed is regulated via caveolin-mediated endocytosis and macropinocytosis.

12: A method for integrated chemical modification of small interfering RNAs (siRNAs), comprising:
incorporating one or more sites of sense strand or antisense strand of siRNA with D- or L-isonucleosides; and
modifying 3′-end of the sense strand and/or the antisense strand of siRNA with peptide-conjugation.

13: A method for integrated chemical modification of small interfering RNAs (siRNAs), comprising:
incorporating one or more sites of sense strand or antisense strand of siRNA with D- or L-isonucleosides; and
using cationic liposome vector to realize the transmembrane transport of the modified siRNAs.

14: A method for integrated chemical modification of small interfering RNAs (siRNAs), comprising:
modifying 3′-end of the sense strand and/or the antisense strand of siRNA with peptide-conjugation; and
using cationic liposome vector to realize the transmembrane transport of the modified siRNAs.
